(19) United States
(12) Patent Application Publication
(10) Pub. No.: US 2012/0095537 A1
(43) Pub. Date: Apr. 19, 2012

(54) Method and Apparatus for Inducing Therapeutic Hypothermia

(76) Inventors: Gregory W. Hall, Los Gatos, CA (US); Daniel Rogers Burnett, San Francisco, CA (US); Daniel P. Seidman, Oakland, CA (US)

(21) Appl. No.: 13/275,189

(22) Filed: Oct. 17, 2011

Related U.S. Application Data
(63) Continuation-in-part of application No. 12/702,165, filed on Feb. 8, 2010.
(60) Provisional application No. 61/393,794, filed on Oct. 15, 2010.
(60) Provisional application No. 61/450,067, filed on Mar. 7, 2011.

(51) Int. Cl. A61F 7/12

(52) U.S. Cl. 607/105

(57) ABSTRACT

Methods and apparatus for delivering therapeutic hypothermia to a patient are provided which may include any number of features. One feature is a hypothermia system comprising a fluid source, a heat exchanger assembly, a catheter in fluid communication with the fluid source, and a pump system configured to infuse hypothermic fluid into a patient cavity and extract hypothermic fluid from the patient cavity. The hypothermia system can infuse and extract fluid automatically from the patient cavity. In one embodiment, the patient cavity is a peritoneal cavity. A safe access device to gain access to the patient cavity is also provided.

### CROSS REFERENCE TO RELATED APPLICATIONS

This application is a continuation-in-part of pending U.S. application Ser. No. 12/702,165, filed Feb. 8, 2010, titled “Method and Apparatus for Inducing Therapeutic Hypothermia”.

This application also claims the benefit under 35 U.S.C. 119 of U.S. Provisional Patent Application No. 61/393,794, filed Oct. 15, 2010, titled “Hypothermia Catheter and System”, and U.S. Provisional Patent Application No. 61/450,067, filed Mar. 7, 2011, titled “Hypothermia Devices and Methods”. These applications are herein incorporated by reference in their entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD OF THE INVENTION

The present invention relates generally to medical/surgical devices and methods pertaining to hypothermia, hyperthermia and normothermia. More specifically, the present invention relates to devices and methods infusing a hypothermic fluid into a cavity of a patient, such as a peritoneal cavity, to induce therapeutic hypothermia.

### BACKGROUND OF THE INVENTION

Hypothermia has been shown to provide distinct medical benefits to stroke and cardiac arrest patients by limiting the size of the infarction and related tissue injury if initiated soon enough and if the level of cooling is significant enough. Both of these limitations, initiation of and depth of cooling, have made practical application of the technology quite challenging particularly in an ambulance or other emergency settings in the field. Initiation of cooling, for example, is a major issue since most technologies require sophisticated machinery that would be difficult to place in ambulance so the patient, at best, receives the hypothermic benefit some time after they reach the hospital. Of the technologies that can be initiated in the field, though, such as cooling blankets, cooling caps, etc., the depth of cooling is a major issue due to surface area limitations, complications (such as intense shivering response) and patient access issues (once the blanket is on, it may be difficult to access the patient).

Thus, there exists a need for improved devices for rapidly producing hypothermia to treat stroke, severe cardiac events and related conditions, particularly in field settings.

### SUMMARY OF THE INVENTION

In one embodiment, a hypothermia system comprises a fluid source containing a hypothermic fluid, a heat exchanger assembly having a heat transfer surface, a heat exchanger module configured to mate with the heat transfer surface of the heat exchanger assembly, a catheter in fluid communication with the fluid source through the heat exchanger module, and a pump system configured to infuse hypothermic fluid into a patient cavity through the catheter and extract hypothermic fluid from the patient cavity through the catheter.

In some embodiments, the patient cavity is a cavity that outside of the vasculature or blood vessels. The patient cavity can be a peritoneal cavity, for example. In some embodiments, the catheter is configured to deliver the hypothermic fluid into the patient cavity. The catheter can also be configured to extract the hypothermic fluid from the patient cavity.

In some embodiments, the heat transfer surface is a thermoelectric surface.

In one embodiment, the heat exchanger module further comprises channels in fluid communication with the fluid source and the catheter. The channels can be heat formed tubes or aluminum tubes in the form of a coil, for example.

In other embodiments, the heat exchanger assembly further comprises a mechanism configured to engage the heat exchanger module in firm contact with the heat transfer surface. The mechanism can be a door, for example.

In some embodiments, the pump system is configured to infuse and extract the hypothermic fluid into and out from the patient without contacting the hypothermic fluid. The pump system can comprise at least one peristaltic pump, for example. In one embodiment, the pump system comprises an infusion pump and an extraction pump.

In another embodiment, a hypothermia system is provided comprising a fluid source containing a hypothermic fluid, a heat exchanger assembly having a heat transfer surface, the heat exchanger assembly configured to receive a heat exchanger module in fluid communication with the fluid source, a pump system configured to receive an infusion line an extraction line both in fluid communication with the heat exchanger module, the pump system configured to infuse and extract the hypothermic fluid into and out from a patient without contacting the hypothermic fluid.

In some embodiments, the heat transfer surface is a thermoelectric surface.

In another embodiment, the heat exchanger assembly further comprises a mechanism configured to engage the heat exchanger module in firm contact with the heat transfer surface. In some embodiments, the mechanism is a door.

In some embodiments, the pump system comprises at least one peristaltic pump. The pump system can comprise an infusion pump and an extraction pump.

In one embodiment, the hypothermia system further comprises a catheter, and the pump system is configured to infuse and extract the hypothermic fluid into and out from the patient through the catheter. The catheter can be an intraperitoneal catheter, for example.

Another embodiment provides a disposable hypothermia administration set, comprising a heat exchanger module configured to mate with a heat transfer surface of a hypothermia machine, a reservoir connector attached to the heat exchanger module and configured to join the heat exchanger module in fluid communication with a fluid source of the hypothermia machine, an infusion line and an extraction line in fluid communication with the heat exchanger module, the infusion and extraction lines configured to mate with a pump system of the hypothermia machine without permitting the pump system to directly contact fluid within the infusion and extraction lines.

In some embodiments, the heat exchanger module further comprises channels in fluid communication with the fluid source and the infusion and extraction lines. The channels can be heat formed tubes, for example.

In one embodiment, the disposable hypothermia administration set further comprises a catheter in fluid communication with the infusion and extraction lines. The catheter can be an intraperitoneal catheter, for example.

In one embodiment, an entry detection device configured to penetrate into a cavity of a patient is provided, comprising an elongate shaft having a lumen extending therethrough and a tissue penetrating tip, and a fluid source in fluid communication with the lumen, the fluid source configured to release a volume of fluid into the cavity when the tissue penetrating tip gains access to the cavity.

In some embodiments, the entry detection device of claim further comprises threads on the elongate shaft.

In other embodiments, the fluid source holds between 5 ml and 60 ml of fluid. In another embodiment, the fluid source holds at least 50 ml of fluid.

In some embodiments, the entry detection device further comprises a lubricious coating on the elongate shaft. In another embodiment, the entry detection device comprises an ultrasound coating. In another embodiment, the entry detection device further comprises a thrombogenic coating.

In some embodiments, the tissue penetrating tip has a diameter between 5 mm and 12 mm.

In one embodiment, the elongate shaft comprises a plastic.

In some embodiments, the entry detection device further comprises a sensor configured to detect release of the fluid from the fluid source into the cavity.

In one embodiment, the entry detection device comprises a taper disposed near a proximal end of the elongate shaft, the taper configured to mate with the fluid source.

In some embodiments, the fluid source is removable from the elongate shaft.

In some embodiments, the fluid source is pressurized. In other embodiments, the release of fluid is passive (e.g., gravity).

A method of gaining access to a cavity of a patient is provided, comprising inserting an entry detection device into the patient, detecting access to the cavity when a volume of fluid drains from the entry detection device into the cavity.

In some embodiments, the volume of fluid is approximately 5 ml to 60 ml. In another embodiment, the volume of fluid is at least 50 ml.

In some embodiments, the detecting step further comprises detecting access to the cavity when a volume of fluid drains at a rate of at least 0.25 in/sec from the entry detection device into the patient cavity. In another embodiment, the detecting step further comprises detecting access to the cavity when a volume of fluid drains at a rate of at least 0.37 in/sec from the entry detection device into the patient cavity.

In some embodiments, the cavity is a peritoneal cavity.

In one embodiment, a peritoneal infusion and extraction catheter is provided, comprising a first lumen and a second lumen, a plurality of extraction ports disposed near a distal portion of the first lumen, the extraction ports having a diameter of approximately 0.035″ to 0.045″ and being spaced approximately 0.2″ from each other, and a plurality of infusion ports disposed on the second lumen, the infusion ports positioned proximally along the catheter from the extraction ports, the infusion ports having a diameter of approximately 0.035″ to 0.045″, or alternatively, up to 0.10″ in diameter, and being spaced approximately 0.25″ from each other.

In some embodiments, a cross-sectional area of the first lumen is two times the cross sectional area of the second lumen. In another embodiment, the cross sectional area of the first lumen is three times the cross sectional area of the second lumen.

In some embodiments, the peritoneal infusion and extraction catheter comprises an integral pressure sensor. The integral pressure sensor can be a fluid column, or, alternatively, the integral pressure sensor can be an electronic pressure sensor.

In some embodiments, the peritoneal infusion and extraction catheter is configured to deliver a hypothermic solution to a patient through the plurality of infusion ports at a rate of approximately 1.3 to 2 liters per minute. In other embodiments, the catheter can deliver a hypothermic solution to the patient at a rate up to approximately 3-4 liters per minute.

In some embodiments, the peritoneal infusion and extraction catheter can further comprise a temperature sensor disposed near the infusion ports, the temperature sensor configured to measure an infusate temperature of a hypothermic solution as it is delivered to a patient.

In one embodiment, the peritoneal infusion and extraction catheter further comprises a weight disposed near a distal end of the catheter. In one embodiment, the weight is magnetic.

In some embodiments, the peritoneal infusion and extraction catheter further comprises additional extraction ports disposed near the distal portion of the second lumen, the peritoneal infusion and extraction catheter further comprising communication ports disposed between the first and second lumens to allow fluid communication between the first and second lumens

In some embodiments, the plurality of extraction ports are patterned so that there are multiple holes at each transverse section of the catheter.

In one embodiment, a method for placing a catheter within a cavity of a patient is provided comprising inserting the catheter having a magnetic tip into the cavity, moving a magnet external to the patient and in magnetic communication with the magnetic tip inside the patient to drag the catheter to a desired location within the cavity.

A method for inducing hypothermia in a patient is provided comprising infusing between 2 and 6 liters of a hypothermic fluid into a patient to reduce a core temperature of the patient at least 3 degrees C. in less than 10 minutes.

In one embodiment, a method for inducing hypothermia in a patient comprises infusing a hypothermic fluid from a fluid source into a cavity of the patient, sensing a change in weight of the fluid source, and stopping delivery of the hypothermic fluid to the patient when the change in weight of the fluid source reaches a predetermined value.

In another embodiment, a method for inducing hypothermia in a patient comprises infusing a first volume of hypothermic fluid into a cavity of the patient at a rate of infusion, upon delivering the first volume of hypothermic fluid into the cavity, extracting the hypothermic fluid from the cavity at a rate of extraction, the rate of extraction being larger than the rate of infusion, and stopping or slowing the extraction of fluid from the cavity when a predetermined volume of fluid remains in the cavity.

In an alternative embodiment, a method for inducing hypothermia in a patient comprises infusing a hypothermic fluid having a temperature less than 32.5° C. into a cavity of the patient to cool a core temperature of the patient, and warming the infused hypothermic fluid within the cavity when the core temperature of the patient reaches a target temperature.

In some embodiments, the target temperature is 32.5° C.

In another embodiment, the infused hypothermic fluid is warmed to match the temperature of the target temperature.

In some embodiments, the infused hypothermic fluid is warmed to a temperature higher than the target temperature.

In another embodiment, the infused hypothermic fluid is warmed until the core temperature of the patient stops decreasing.

In one embodiment, a method for inducing hypothermia in a patient is provided, comprising infusing a fluid into a cavity of the patient at an infusion rate, extracting the fluid from the patient at an extraction rate equal to the infusion rate, cooling the fluid, and infusing the fluid back into the cavity of the patient.

In another embodiment, a method of automatically detecting and removing an obstruction in a hypothermia system during the delivery of therapeutic hypothermia comprises infusing a hypothermic fluid into a patient to induce hypothermia, sensing a system parameter of the hypothermia system, and reversing a direction of flow of the hypothermic fluid when the sensed system parameter indicates an obstruction in the hypothermia system.

In some embodiments, the system parameter is a temperature of the hypothermic fluid.

In other embodiments, the system parameter is a pressure of the hypothermic fluid.

In additional embodiments, the system parameter is a weight of fluid accumulation in a waste bag of the hypothermia system.

Sometimes, the method further comprises sensing a patient parameter and reversing a direction of flow of the hypothermic fluid when the sensed patient parameter indicates an obstruction in the hypothermia system.

In one embodiment, a method for inducing hypothermia in a patient comprises cooling a fluid with a heat exchanger to an infusion temperature, infusing the cooled fluid into a cavity of the patient in response to increases in patient temperature over a target value. Fluid is extracted by either actively pumping or passively draining from the cavity.

In one embodiment, a method for inducing hypothermia in a patient comprises sensing a temperature of a temporal artery to determine a core temperature of the patient, and controlling the infusion and extraction of a hypothermic fluid into the patient based on the sensed temperature.

In one embodiment, a method for inducing hypothermia in a patient comprises sensing a temperature of a tympanic membrane to determine a core temperature of the patient, and controlling the infusion and extraction of a hypothermic fluid into the patient based on the sensed temperature.

In some embodiments, the sensing step further comprises sensing the temperature of the tympanic membrane with an infrared sensor in an ear canal of the patient.

In one embodiment, a method for inducing hypothermia in a patient comprises sensing a temperature of a supero-medial orbit of an eye to determine a core temperature of the patient, and controlling the infusion and extraction of a hypothermic fluid into the patient based on the sensed temperature.

In some embodiments, the sensing step further comprises sensing the temperature of the supero-medial orbit of the eye with an infrared sensor.

In one embodiment, a method for inducing hypothermia in a patient comprises sensing a temperature of an abdominal wall to determine a core temperature of the patient, and controlling the infusion and extraction of a hypothermic fluid into the patient based on the sensed temperature.

In one embodiment, a method for inducing hypothermia in a patient comprising sensing a temperature of a tympanic membrane to determine a core temperature of the patient, and controlling the infusion and extraction of a hypothermic fluid into the patient based on the sensed temperature.

In one embodiment, a method for inducing hypothermia in a patient comprises sensing a temperature within a subcutaneous tissue to determine a core temperature of the patient, and controlling the infusion and extraction of a hypothermic fluid into the patient based on the sensed temperature.

In one embodiment, a method for inducing hypothermia in a patient comprises sensing a temperature of within muscle tissue to determine a core temperature of the patient, and controlling the infusion and extraction of a hypothermic fluid into the patient based on the sensed temperature.

In one embodiment, a method for inducing hypothermia in a patient comprises sensing a temperature of within layers between tissue types (fascia) to determine a core temperature of the patient, and controlling the infusion and extraction of a hypothermic fluid into the patient based on the sensed temperature.

In another embodiment, a method of inducing therapeutic hypothermia in a patient is provided, comprising a) infusing a fill volume of hypothermic fluid into a patient cavity to cool the patient at a first cooling rate until reaching a first end condition, b) after step a), removing hypothermic fluid from the patient cavity while infusing hypothermic fluid into the patient cavity to cool the patient to a first set point temperature at a second cooling rate, and, c) after step b), removing hypothermic fluid from the patient cavity while infusing hypothermic fluid into the patient cavity to cool the patient to a second set point temperature at a third cooling rate, the third cooling rate being slower than the second cooling rate.

In some embodiments, the first end condition comprises a sensed pressure in the cavity reaching a pressure limit.

In some embodiments, the pressure limit is greater than 14 mm Hg.

In other embodiments, the first end condition comprises infusing a fill volume of approximately 2 liters, 3 liters, 4 liters, 5 liters, or 6 liters.

In some embodiments, the first set point temperature is approximately 34° C. In some embodiments, the second set point temperature is approximately 32.5° C.

In some embodiments, the first cooling rate is approximately 12° C./hr. In another embodiment, the second cooling rate is approximately 6° C./hr. In some embodiments, the third cooling rate is approximately 1° C./hr.

In one embodiment, the hypothermic fluid is infused at a rate of approximately 1-2 liters per minute. In some embodiments, in step b), the hypothermic fluid is infused and removed at the same rate. In additional embodiments, in step c), the hypothermic fluid is infused and removed at the same rate.

In some embodiments, in step b), the hypothermic fluid is infused and removed at a rate of approximately 0.1 to 0.3 liters per minute. In other embodiments, in step c), the hypothermic fluid is infused and removed at a rate of approximately 0.1 to 0.15 liters per minute.

In some embodiments, the hypothermic fluid infused in step b) is colder than the hypothermic fluid infused in step c). In another embodiment, the hypothermic fluid infused in step b) is a fresh hypothermic fluid from outside of the patient. In an additional embodiment, the hypothermic fluid infused in step c) is a recycled hypothermic fluid from inside of the patient. In some embodiments, the recycled hypothermic fluid is cooled prior to being re-infused into the patient.

In some embodiments, the method further comprises detecting an obstruction, and reversing a flow direction of the hypothermic fluid to remove the obstruction.

In another embodiment, the method further comprises the step of d) draining the hypothermic fluid from the patient.

In some embodiments, in step d), the hypothermic fluid is drained at a rate of approximately 0.1 liters per minute.

In one embodiment, if the second cooling rate of step c) is below a desired rate, increasing the cooling rate.

Some embodiments further comprise adjusting the first and second cooling rates in real time by adjusting at least one of a pump speed, a cooling/warming power, or a fluid source based on a treatment parameter.

In some embodiments, the treatment parameter comprises a time of treatment. In other embodiments, the treatment parameter comprises a cavity pressure. In additional embodiments, the treatment parameter comprises a core temperature. In yet another embodiment, the treatment parameter comprises the fill volume.

In some embodiments, the fill volume is determined based on an amount of cooling energy needed to cool the patient.

The method can further comprise the steps of d) infusing additional hypothermic fluid beyond the fill volume into a patient cavity and surpassing the pressure limit so as to cool the patient faster than the first, second, or third cooling rates; and e) removing the additional hypothermic fluid after a pre-determined time period so the pressure limit is not surpassed beyond a tolerable limit.

In some embodiments, the method further comprises maintaining therapeutic hypothermia in the patient with steps a)-c) for at least 24 hours.

In one embodiment, the method further comprises warming the patient back to normothermic temperature.

In some embodiments, a rate of warming is controlled to a specified rate of warming. In one embodiment, if the patient is warming too quickly, a cooler hyperthermic solution is circulated in the patient cavity to arrest a warming rate. In another embodiment, if the patient is warming too slowly, a warmer hyperthermic solution is circulated in the patient cavity to increase a warming rate. In some embodiments, if the patient is warming too quickly, a pump rate of hyperthermic solution can be lowered to arrest a warming rate. In other embodiments, if the patient is warming too slowly, a pump rate of hyperthermic solution can be increased to increase a warming rate.

A method of inducing therapeutic hypothermia in a patient is provided, comprising a) infusing a fill volume of hypothermic fluid into a patient cavity to cool the patient at a first cooling rate until reaching a first end condition, and b) after step a), removing hypothermic fluid from the patient cavity while infusing hypothermic fluid into the patient cavity to cool the patient to a set point temperature at a second cooling rate.

In some embodiments, in step b), the infused hypothermic fluid is drawn from a remote fluid reservoir. In other embodiments, in step b), the removing and infusing hypothermic fluid steps are performed at the same fluid flow rate. In some embodiments, the fill volume is maintained at the same level during step b).

In some embodiments, the set point temperature is approximately 34° C. In other embodiments, the set point temperature is approximately 32.5° C.

### BRIEF DESCRIPTION OF THE DRAWINGS

FIGS. 1A and 1B are schematic illustrations of a therapeutic hypothermia system.

FIG. 1C is a close up view of a catheter connection to a hypothermia system.

FIGS. 2A and 2B are illustrations of a patient cavity access device.

FIGS. 3A-3C are schematic views of an infusion and extraction catheter.

FIGS. 4A-4C are schematic illustrations of a therapeutic hypothermia system.

FIGS. 5A and 5B are flowcharts describing methods of treatment.

FIGS. 6A and 6B are plots showing the patient temperature, infusate temperature, and infusate volume of fluid in the patient during the treatment.

FIGS. 7A-7B show plots of a patient respiratory cycle during treatment.

### DETAILED DESCRIPTION OF THE INVENTION

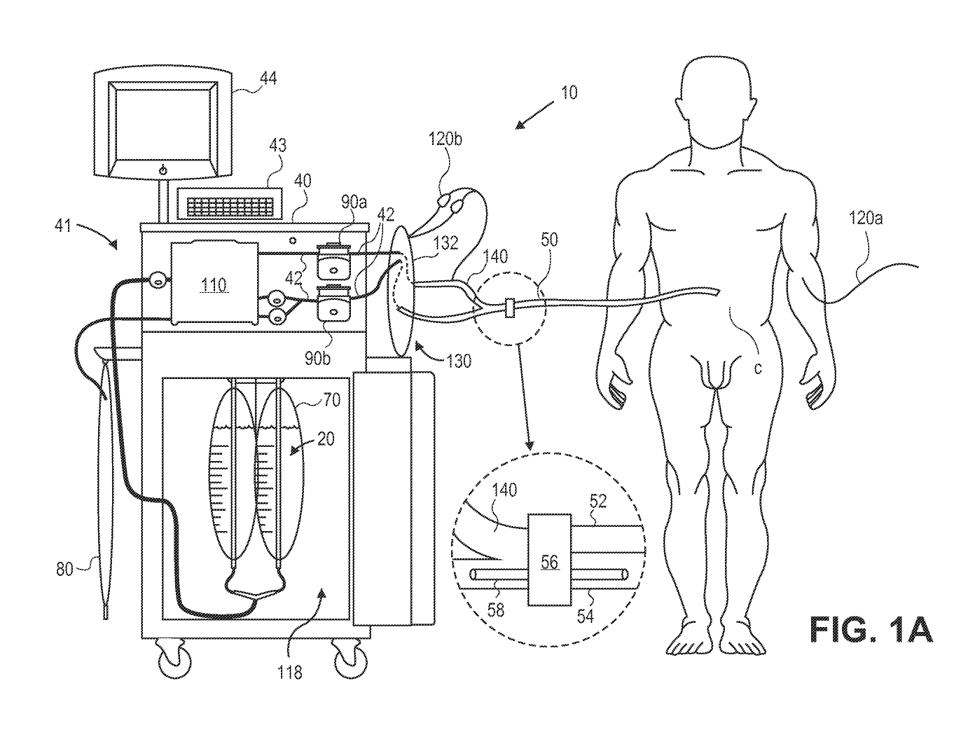

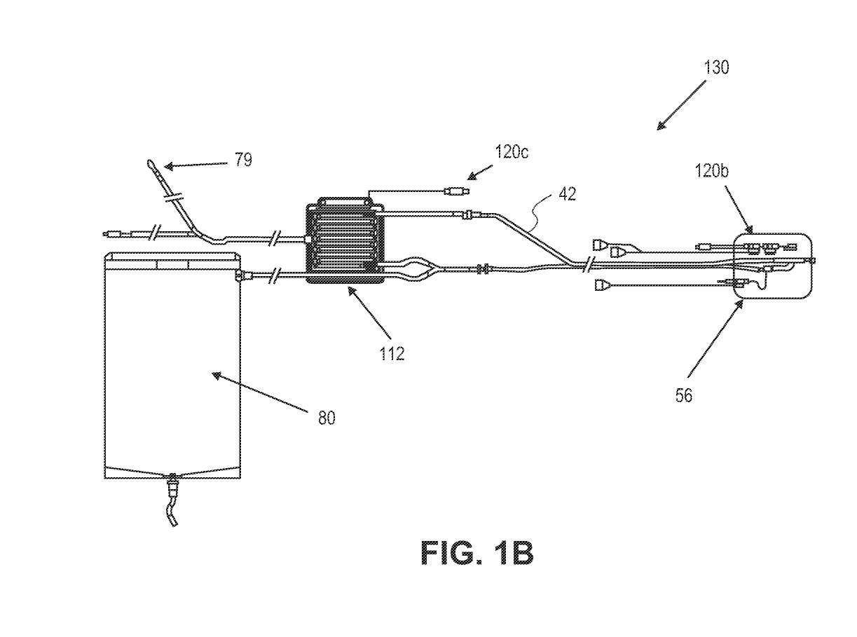

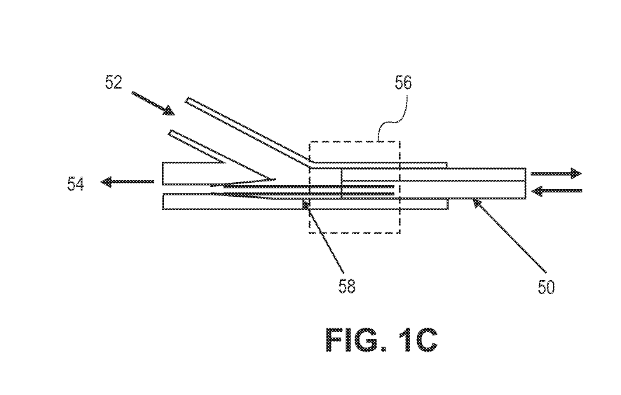

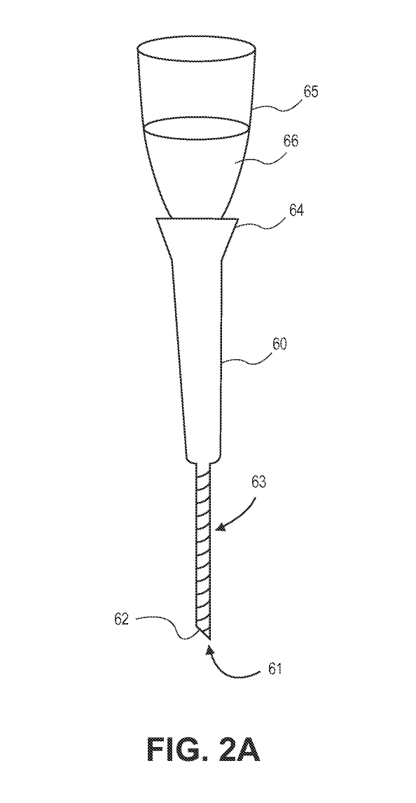

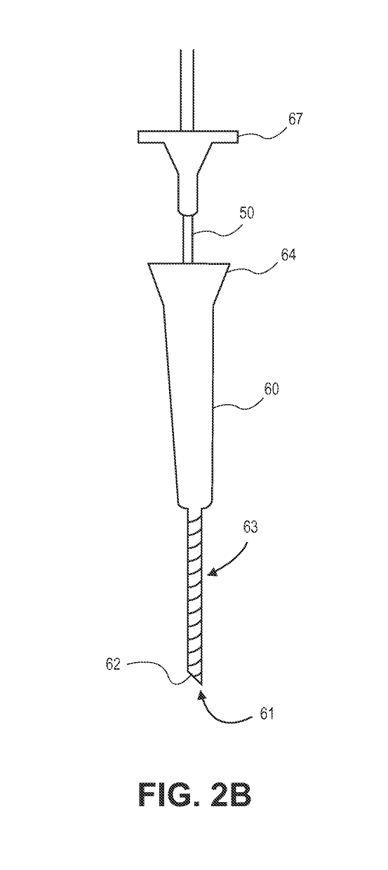

FIGS. 1A-1B illustrate an embodiment of a system 10 for the delivery of hypothermic or other fluid 20 to a peritoneal or other tissue cavity C. The system can comprise a main unit 40, controller 41, a catheter 50, an access device 60 (shown in FIGS. 2A-2B), a fluid source or fluid reservoir 70, a waste fluid container 80, a pumps 90a and 90b, a heat exchanger assembly 110, one or more sensors such as temperature sensors 120a or pressure sensors 120b, and a Lavage Administration Set (LAS) 130 (Shown in more detail in FIG. 1B). In various embodiments, system 10 can be used to deliver fluids to a number of body cavities such as the peritoneal cavity, the pleural cavity, vagina, digestive tract, nasal cavity, cerebrospinal fluid cavity, and like structures, as well as to various vascular structures. Furthermore, a therapeutic or hypothermic fluid can be delivered to the patient cavity to achieve therapeutic hypothermia, post hypothermic warming, hyperthermia, resuscitation, blood pressure management, controlled thermal necrosis, and other related treatments. For ease of discussion, system 10 will be referred to as a peritoneal circulation or hypothermia (PH) system 10 and the cavity will be the peritoneal cavity. However this is for illustrative purposes and it should be appreciated that other uses and application sites are equally applicable. For example, embodiments can be readily configured for use in the pleural cavity through selection of dimensions, shape, materials etc. In some embodiments, access to a cavity may be through external or internal means. The peritoneal cavity may be accessed through the abdominal wall, the stomach wall, the bladder wall, the rectum wall, or other approaches.

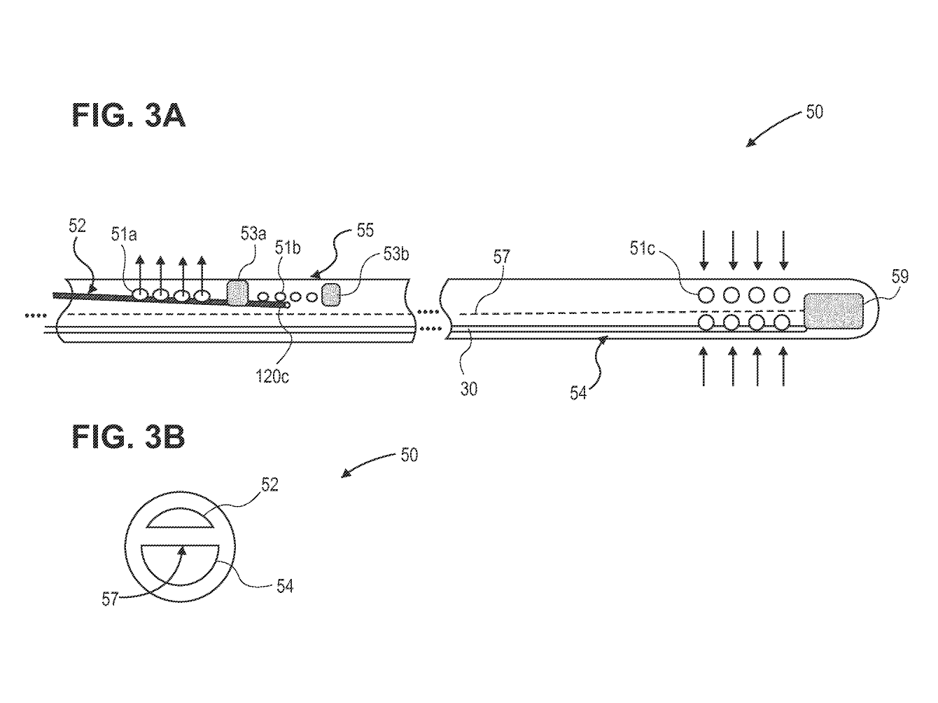

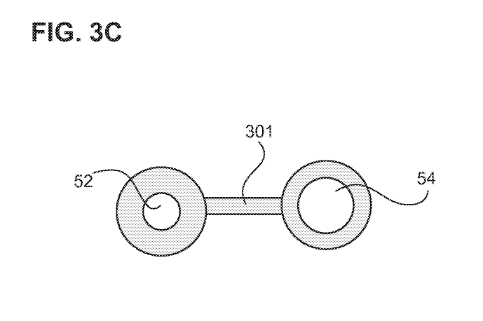

Catheter 50 can be a single or multi-lumen catheter. In the embodiment of FIGS. 1A and 3A-3B, the catheter is a dual lumen catheter, comprising an infusion lumen 52 and an extraction lumen 54. A third lumen for pressure-measurement can reside in the infusion lumen to provide fluid communication with external pressure sensors. Alternative embodiments include one or more pressure sensor and/or temperature sensors disposed within either the infusion lumen or the extraction lumen. The proximal end of infusion catheter 50 can be coupled to unit 40 of the hypothermia system 10 through the LAS 130 and either fluid or electrical sensor connections. The distal end of the catheter is configured to be positioned within the peritoneal or other cavity of the patient so as to infuse fluid 20 into the cavity.

In some embodiments, the catheter and LAS may be constructed such that the infusion lumen has a smaller internal diameter and thicker wall than the extraction lumen. This may ensure that the infusate travels faster through the patient lines with less heat absorption from ambient air, the patient, or an adjacent lumen. The extraction lumen can be larger with a thin wall because heat loses from the extraction line to the ambient room temperature can aid in increasing cooling power. In the embodiment shown in FIG. 3C, the infusion and extraction lumens may be spaced apart with a web, as shown in F. In some embodiments, the web may be long (1″ or so) to prevent heat transfer between the infusion and extraction lines. The web can also serve to hold a pressure lumen or electrical wires for tidy packaging of the device components. Wires and/or tubes can be woven through the web at spacing to prevent tangling and damage. In this design, the internal diameter of the extraction lumen can be nominally ¼″ and the internal diameter of the infusion lumen can be nominally ⅛″.

Referring now to FIG. 1B, LAS 130 can comprise a hub 140, patient lines 42, reservoir connector 79, pressure sensors 120b, infusate temperature sensor 120c, and heat exchanger module 112. Waste fluid container 80 is also shown in FIG. 1B, but is not necessarily a part of the LAS 130. As shown in FIGS. 1A-1B, the catheter 50 can mate with hub 140 of LAS 130 at connector 56. The hub can then be coupled to patient lines 42, which are attached to the heat exchanger module 112 of heat exchanger assembly 110. The patient lines may be made from a single, dual-lumen extrusion (Double D, or tangent circles, or circles with a septum for example). Alternatively the patient lines may be separated by an air space and held together with clips, to prevent heat transfer between the infusion line and extraction line. The infusion patient line may be insulated or have a thicker wall to prevent cold or warm fluid changing temperature towards ambient temperature as it travels from the heat exchanger to the patient. Generally, insulation on the extraction patient line is not required because thermal radiation from the extraction line to room temperature assists with maintaining a cold patient temperature.

Pressure sensors 120b can attach to the hub 140 or directly to the catheter to receive pressure information from the catheter. The pressure sensors can detect pressure information from within a patient cavity, such as intraperitoneal pressure, or the pressure sensors can detect a pressure of the fluid or infusate 20 within the system 10. In some embodiments, separate pressure sensors can detect both patient cavity pressure and fluid pressure within the system, for example. Further details of the pressure sensors will be described below.

A soft cover may be attached to the hub and sealed around the hub, catheter, and pressure lumen connection during device set-up. The soft cover can serve to prevent tampering with the device during use and can reduce irritation of the patient skin.

The LAS 130 can be packaged in a sterile pouch (or tray or similar) to keep all components sterile prior to connection to the catheter and the main unit 40. The LAS can be removed from its packaging for installation to the main unit 40 with the patient lines, pressure sensors, and hub protected by a secondary sterile/protective barrier 132. Once access to the patient has been achieved, a user can open the sterile pouch 132 to connect the patient lines 42 to catheter 50 at connector 56.

The LAS 130 can also include a recirculation cap attached to the hub prior to connecting the LAS to the catheter. The recirculation cap may allow an “auto-prime” sequence to flush air from the LAS and/or to initiate lower temperatures of the LAS prior to patient treatment. The “auto-prime” sequence will be discussed in more detail below.

Referring now to FIGS. 1A and 1C, when hub 140 is connected to catheter 50 at connector 56, a first branch of the hub 140 can couple the infusion lumen 52 to fluid sources 70 through the patient lines 42, and a second branch of the hub can couple the extraction lumen 54 to either the waste fluid container 80 or the heat exchanger 110 through the patient lines. An optional tube 58 may be inserted into one of the catheter lumens, such as the extraction lumen 54, to aid in aligning each branch of the hub with the appropriate catheter lumen. The tube 58 can snugly fit within a conical taper, a lip seal, or an o-ring in the hub to seal the hub and catheter together. Specific hub alignment may, however, not be required. The tube can also extend beyond a junction in the hub (i.e., where the two branches of the hub join) to prevent any waste carried by the extraction lumen from being pumped back into the patient through the infusion lumen. The tube could optionally be mounted in the hub to serve the same purpose described above. In one embodiment, the tube comprises aluminum or another MRI compatible material.

In some embodiments, the connection between the catheter 50 and the remainder of the system (e.g., connector 56) is done with a single motion that aligns the infusion, extraction, and pressure lumens with corresponding lumens in the LAS hub. The user can push the catheter into the receiving end of the connector until a certain force is reached or until there is an audible “click” or tactile feedback. This tactile feedback can be provided by a detent feature, such as an extrusion passing by an O-ring or by a feature bottoming out into a tapered shaft. In one embodiment, the connection can complete an electrical circuit, providing a signal to the control system to indicate that the catheter has been connected properly. For example, connector 56 can have a resistor of a particular value to indicate an identifying piece of information (e.g., lot #, model #, calibration, etc).

In other embodiments, the connector 56 can be any kind of catheter connecter as known in the art, such as a Luer lock connector, a Tuohy Borst adapter, a flare connector, a hub connector, a snap fit and/or quick disconnect, a lip seal, or a circumferential clamp, for example. The connector may further include an O-ring to seal to the outer diameter of the catheter. The connector may also include electrical connections for connectivity of components such as but not limited to pressure, sensors, temperature sensors and video connection.

A temperature or other patient sensor 120a can be connected to the patient at an IV or other site to measure the patient's temperature. In FIG. 1A, temperature sensor 120a is shown connecting to the patient's arm. However, in another embodiment, the temperature sensor 120a measures a temperature of the esophagus. Such temperature measurement may be external or internal to the patient. After the catheter 50 is positioned at the desired body cavity site, the infusion (and removal) of fluid 20 can be initiated either under manual or automated control. The user can see various data (e.g., patient temperature) on displays 44 and make one or more adjustments using buttons or other user interface 43, or place the unit in an automated mode.

In various embodiments, fluid 20 comprises a solution 20 for the delivery of a medical treatment such as hypothermic or resuscitative treatment.

For ease of discussion, fluid 20 will be referred to as solution 20 or as infusate 20. Suitable solutions 20 can comprise various saline solutions (e.g., lactated ringers solution), various peritoneal dialysis fluids including nutritive based peritoneal dialysis fluid (e.g., those containing dextrose and other sugars), fluorocarbon solutions configured for oxygen transport and artificial blood solutions known in the art. In some embodiments, heparin can be added to the fluid to reduce the likelihood of protein and blood deposition in the abdominal cavity during treatment. For aqueous embodiments, the solution can also include one or more freezing point depression compounds (e.g., NaCl) allowing the solution to be cooled below the freezing point of water to allow for faster cooling when so desired. In some embodiments, the solution may comprise an ice slurry.

Also, solution 20 can contain one or more medicaments for treatment of myocardial infarction, cardiac arrest or other severe cardiac condition, stroke, shock, reperfusion injury or other medical conditions. Specific families of medicaments can include vasoconstrictors, hemolytic compounds (e.g., TPA, streptokinase and like compounds), anticoagulants, coagulants, calcium channel blockers, antibiotics, manitols. Also in specific embodiments, solution 20 can be configured to have resuscitative effects for treatment of heart attack, stroke, or severe blood loss. It can also have various agents known in the art for treatment of reperfusion injury. The delivered amount of a particular medicament can be titrated to the patient's weight and condition, with titration controlled manually or by a drug delivery module resident within controller 41. Also, the dose of particular compounds can both be delivered as bolus with the initial bolus of hypothermic solution and also on a continuous basis. The delivery rate of a particular medicament or group of medicaments can also be controlled responsive to the patient's temperature, blood pressure, heart rate or other vital sign monitored manually, by system 10, or by other monitoring methods.

Solution 20 can also comprise oxygenated solutions such as oxygenated fluorocarbon solutions that can be configured to deliver sufficient oxygen to tissue (by gas exchange with peritoneal or other surrounding tissue) to at least partially meet the oxygen demands of the body. Fluorocarbon solutions can be pre-oxygenated or can be oxygenated in reservoir 70 or outside of it using oxygen gas sources described herein. Solution 20 can also include contrast media to allow for imaging by x-ray, MRI, ultrasound, and other imaging modalities known in the art.

The main unit 40 will typically be contained in a frame or housing and will frequently include a handle for portability, which can be disposed at any point on the frame. While unit 40 can be a standalone unit, it can also be configured to be readily attached to and detached from other components of system 10, such as reservoir 70, waste container 80, pumps 90a and 90b, and refrigerator 118 as is discussed herein.

Displays 44 and user interfaces 43 can comprise a console face or a console device. The console face can be permanently attached to the unit; however it may be pivotal in multiple directions to allow viewing from different angles. In particular embodiments, it may also be removable, functioning as remote console that wirelessly communicates with the main unit. In use, such wireless embodiments allow the user to operate the system from any position around the patient, or even to do so remotely. This provides the user with greater flexibility and ease of use in both setting up and operating the system, including faster response time in making system adjustments. For example, if the user sees that the patient requires immediate attention due to fallen blood pressure, blood oxygen saturation, etc. or even cardiac arrest, they can make an immediate adjustment to the system using the remote console rather than having to rush to reach the control unit. User interface 43 can be a keyboard, a remote console, a GUI, a joystick, buttons on the display 44 or on another device, or any other known user interface.

Content of the user interface may include instructional diagrams or videos of how to operate the device, real time data of critical parameters, pictorial representations of the system with graphical indicators conveying where a problem is, plots of the temperature and power usage time histories. Power usage can be used as an indicator of fever or infection in the patient since body remains constant. It may also alert the user indicate system or patient issues.

The main unit 40 further comprises a controller 41. In many embodiments, the controller can be configured to automatically control one or more parameters related to a hypothermic or other treatment regimen, for example, infusate temperature, body temperature, infusion and extraction rates, infusion and removal pressures, total volume of fluid infused and extracted, and similar parameters. It should be appreciated that controller 41 can also be configured to perform a variety of operations including communicating with external devices including devices linked over the Internet; wireless peripheral devices; data operations; and various power management functions. The system may also have the ability to read data or other identifiers included in the peripheral devices through means such as resistance coding, EPROM, bar code or other identifiers.

Controller 41 can include one or both of analog or digital circuitry for performing its control operations. The controller will also typically be configured to receive one or more inputs, such as from sensors. Typically, the controller will include a computer processor which is configured to execute one or more electronic instruction sets contained within a software module, which can be stored in memory onboard unit 40. Additionally, controller 41 can be configured to be programmed by the user (e.g., using the user interface or by an external device such as a wireless device) to allow for manual control of one or more operations of system 10 (e.g., infusion rate).

Sensors can be configured to measure a variety of physical properties related to the use of system 10 and the patient. Accordingly, they can comprise a variety of biomedical sensors known in the art, including patient temperature sensors 120a, pressure sensors 120b, infusate temperature sensors 120c, force sensors, flow sensors, pH sensors, oxygen and other gas sensors (e.g., CO₂), acoustic sensors, piezoelectric sensors, and the like. Additionally, sensors can be used to monitor the pressure of cerebral spinal fluid. Suitable temperature sensors can include thermisters, thermocouples, optical sensors, and like devices. In one embodiment, the temperature sensors can be temporal artery sensors. The temporal artery sensors can be attached to patient's skin over the temporal artery to monitor patient temperature. In another embodiment, the temperature sensors can be a wand that is swiped across the temporal artery to measure the patient temperature. The temperature sensors can act as an input into the system, for example. Any of the sensors described herein can communicate wired or wirelessly with the system, such as by Bluetooth, Wifi, RF, infrared, or any other suitable wireless communication protocol. Yet another embodiment is comprised of an array of temperature sensors adhered to the patient's skin in the area of the temporal artery. Either a microprocessor on the sensor array or in the controller selects and possibly calculates the temperature reading most reflective of core temperature. Other temperature sensor methods may include a means to measure through any patient cavity, subcutaneous devices or ingested devices. Another embodiment involves using an infrared sensor in the ear canal to measure tympanic membrane temperature. Alternatively, infrared radiation from the external surface of the supero-medial orbit of the eye (medial corner of the eye) may be used as an indication of core temperature.

Temperature measurement indicative of core temperature can also be obtained by measuring temperature within soft tissues outside of the peritoneal cavity, such as within the abdominal wall, mammary duct or other locations. Alternatively, intramuscular temperatures could be used from other soft tissue locations and muscles groups, particularly those of the thorax, although other locations are being considered.

The system can monitor the volume of fluid within the system and patient by use of one or more volume sensors. Volume of fluid may be detected by the weight of the fluid with a load cell, strain gauge, etc. or by measuring the height of the fluid meniscus in a vessel of known volume. In one embodiment, a single load cell is used to measure both waste and fresh infusate volumes together, although having separate load cells for waste and fresh infusate are currently preferred.

Suitable pressure and force sensors can include strain gauges, electronic pressure sensors, solid state pressure sensors, fluid column pressure sensors, cerebral spinal catheters, air column pressure sensors, and MEMS based strain gauges. Suitable flow sensors include electromagnetic flow sensors, rotary encoder flow sensors, and aneometric flow sensors known in the art or by using infusion line pressure as an indicator of flow. One or more sensors can also include RFID tags or like devices so as to be able to wirelessly signal an input to controller 41 or another instrument. The RFID tags can be used, for example, to communicate patient treatment parameters or patient treatment history to another instrument if the patient is moved or transported to another location. The RFID can also be used to tag solution as used to prevent reuse. Another use of RFID is to store information pertaining to sensors on the catheter such as calibration factors, serial number, lot number, etc. and transfer that information to the controller. Calibration factors may also be stored in a bar code label that is read by the controller or an EPROM chip.

In one embodiment, the controller can receive a blood pressure signal from the patient and adjust the treatment to ensure sufficient abdominal perfusion pressure. Sufficient perfusion pressure is necessary to supply abdominal organs with oxygen and nutrients. The perfusion pressure of an abdominal organ is measured as the difference between Mean Arterial Pressure (MAP) and Intra Abdominal Pressure (IAP). Abdominal pressure could be automatically adjusted to maintain an acceptable level of abdominal perfusion pressure by adjusting the volume and/or pressure within the patient. The system can also alert the user that either the patient blood pressure is too low or the patient may be shivering, thus increasing peritoneal cavity pressures due to abdominal muscle contractions.

In many embodiments, catheter 50 is configured to be advanced into a tissue cavity through use of an access device 60. Access device 60 can be configured to penetrate a distance through the tissue layers surrounding the desired cavity. When the access device is used to gain access to the peritoneal cavity, for example, it will typically penetrate the anterior aponeurosis, the rectus muscle, the posterior aponeurosis, the preperitoneal fat, and finally the peritoneum. The catheter 50 can then be advanced through a lumen 61 of the access device into the peritoneal or other cavity as is described herein. In this respect, access device 60 functions as a port for the introduction and advancement of catheter 50 into tissue. When gaining access to the peritoneal cavity, the catheter is typically advanced towards the patient's right hip or possibly in a pure lateral direction to ensure placement of the distal end of the catheter deep within the posterior aspects of the peritoneal cavity. Other directions of catheter placement can work as well. Raising or “tenting” of the tissue may also increase accessibility laterally above organs or subcutaneous tissues.

Referring to FIGS. 2A-2B, in various embodiments, access device 60 will typically include at least one lumen 61 extending therethrough and a tissue penetrating distal end 62. Lumen 61 can have an inner diameter sized to accommodate a variety of different sized infusion catheters 50 and in various embodiments can range from about 0.1 to 0.5 inches, though other sizes are also contemplated. The distal end can have a variety of tissue penetrating shapes including a trocar tip shape as is shown in FIGS. 2A-2B. In one embodiment, the distal end can also include a built-in skin incision component, such as a scalpel, to make an initial incision in the patient.

Access device 60 can be a surgical port device, trocar or other surgical access device known in the art. Blunt dissection through soft tissues is preferred since it is common for patients to be on anticoagulant medications. In a one embodiment, as illustrated in FIGS. 2A-2B, access device 60 can be a threaded access port or threaded trocar. In some embodiments, the access device can have a diameter of 5 mm to 10 mm, but smaller sizes are contemplated, such as those ranging from 0.5 mm to 5 mm in diameter. One example is a threaded access device with a 0.5 mm size, which could be used allow a guidewire to gain access to a body cavity. A catheter could then be fed over the guide wire, as known in the art.

As shown in FIG. 2A, the access device 60 can include threads 63, which allow a physician to gain access to a body cavity quickly and safely with rotational force, rather than with axial force which risks over-insertion and puncture damage to delicate organs and tissues. The threads 63 can also include a lubricious coating, for example, to ease insertion into tissue, or an ultrasound coating, to aid with visualization of the device during insertion. In another embodiment, the access device can be filled with a solution comprising microbubbles for entry indication using ultrasound visualization. Additionally, the access device or catheter can include a thrombogenic coating to ensure blood clot in the tissue tract. Alternatively, the access device may be threaded into tissue using a gel of thrombogenic material such as chitosan to both lubricate access and arrest bleeding within the access tract.

Due to the varying thicknesses of the sub-dermal tissue layers from patient to patient, a method of knowing when a body cavity is accessed by the access device is desired. One technique is called the “drop test,” and involves inserting a Veress needle into a body cavity, such as the peritoneum, and placing a drop of water or saline on the open end of the Veress needle with the abdominal wall elevated. If the needle is positioned correctly, the solution should disappear down the shaft into the cavity. However, this test can sometimes be falsely positive or falsely negative, for example, such as when an access device strips the peritoneal membrane from the posterior rectus sheath to create a preperitoneal space.

One embodiment of the invention includes a method for accurately determining access to a body cavity with an access device. Access device 60 can include a taper 64 that comprising a flange or lip that protrudes from the outer walls of the access device and is positioned close to the proximal end of the access device. To accurately detect access to a body cavity, such as a peritoneal cavity, a fluid holder 65 containing a volume of fluid 66 can be inserted into the taper 64 of the access device during insertion into the patient. When the body cavity is accessed, a large volume of fluid (typically a minimum of 5 mL but up to 60 mL) will drain from the fluid holder into the patient cavity. Since the user is looking for a large volume of fluid to drain from the fluid holder, instead of the small volume used with the “drop test,” this method can provide a more accurate indicator of proper cavity entry.

A typical volume of fluid in the entry indicator is 50-60 ml, but other volumes are being considered. The volume needs to be sufficient that peeling of the peritoneal lining from the posterior rectus sheath and other cavities formed by the access process do not generate a false indication of entry. The fluid holder can optionally include a sensor, such as an ultrasonic sensor, to automatically detect drainage of fluid when the access device reaches the peritoneum. In another embodiment, the fluid in fluid holder 65 can be pressurized, instead of relying on gravity, to allow the same entry detection if the access device is inserted in a horizontal or “upside down” fashion. The fluid holder is typically transparent so the user can see the rapid drop in fluid level upon access into the cavity. The fluid holder may have markings on the outside to serve as a reference points that aid in detecting motion of the fluid meniscus. In one embodiment, the meniscus can travel between approximately 0.25 and 0.37 in/sec or more when entry is detected to indicate fluid entry. This speed has been determined to be sufficient for distinguishing actual cavity entry vs. artifact.

As shown in FIG. 2B, in embodiments where the access device is left in the patient cavity during treatment, taper 64 of the access device can further interact with a depth sealer 67 located on the catheter 50. The taper may include an elastomeric seal (O-ring), duck bill valve, or other feature that forms a fluid-tight seal with the depth sealer. The depth sealer can have an outer shape that fits within and seals to the inner diameter of taper 64. In addition to sealing the catheter to the access device, the depth sealer also ensures that the catheter is positioned properly with the patient cavity. For example, when the access device is properly inserted into the peritoneum, as discussed above, the fluid holder 65 can be removed. Next, the catheter can be inserted through lumen 61 of the access device until depth sealer 67 engages with taper 64. Since the distance from the tip 62 of the access device to the taper 64 is known, the sealer can be placed along the catheter to position certain features of the catheter, such as infusion ports or apertures 51a, a known or optimal distance from the peritoneal wall in the cavity. It should be understood that many embodiments where the access device is removed once the catheter is inserted into the patient, the catheter will not have a depth sealer device. However, in one embodiment, the access device is a break-away design that does not need to slide off the catheter proximally, so a depth-sealer can still be used. Other sealing techniques may also be employed such as simple tissue pressure against the catheter or other accessory items such as bandages, adhesives, suturing or compression seals.

The catheter 50 will now be discussed. In various embodiments, the catheter can be configured to be positioned in the peritoneal or other tissue cavity and deliver fluid to the cavity for a hypothermic or other medical treatment discussed herein. Typically the catheter will be advanced through an access device 60, which can be inserted to a controlled depth into the abdominal or other tissue wall. The catheter 50 can be configured to be advanced by itself or through use of an advancement member 30, discussed herein, which can be reversibly positioned in a lumen or lumens of the catheter, and acts to increase the pushability or column stiffness of the catheter. Another embodiment involves using a magnet or electromagnet outside the patient to drag a magnetic catheter tip within the patient to the desired location.

The catheter can have a length ranging from 20 cm to 200 cm (though other lengths are being contemplated) to allow for connection to unit 40 at varying distances from the patient. Smaller lengths can be used for a pediatric application. The outer diameter of the catheter can be sized for advancement through standard surgical port access devices and, in varying embodiments, can range from about 0.1 to 1 inch, though other sizes are also contemplated depending upon the target tissue site. For example, smaller sizes can be used for accessing the pleural cavity as well as for pediatric applications. Various embodiments of the catheter can include insertion depth indicia and or radio-opaque/echogenic markings in order to assist in determining insertion depth, either visually or under image guidance (e.g., fluoroscopy, ultrasound, etc.).

Catheter 50 is preferably a single or monolithic extrusion catheter, and can include at least one lumen or chamber extending all or a portion of its length. In FIGS. 3A-3B, the catheter includes an infusion lumen 52 for infusion of solution 20, and an extraction lumen 54 for both removal and infusion of the solution. Additional lumens are also contemplated. Typically, the lumens will be D-shaped or crescent shaped, as shown in FIG. 3B, but can also be round or oval shaped. When the lumens are D-shaped, they can be formed with a wall or web 57 separating the lumens. A round cross section is typical to facilitate sealing of the tissue tract around the catheter after catheter placement.

In various embodiments, the inner diameters and of lumens 52 and 54 can range from about 0.05 to about 0.5 inches though other diameters are also contemplated. Desirably, infusion lumen 52 has a sufficient inner diameter to be able to infuse 2 to 6 liters of hypothermic solution at a rate of 1.3-2 liters per minute to reduce the patient's body temperature by at least about 3° C., or more preferably, in ten minutes or less via heat exchange with peritoneal tissue using pressures less than 3 atmospheres and more preferably less than 1 atmosphere. The extraction lumen is also desirably configured to remove similar volumes of fluid in similar time periods. The relative cross sectional area of the infusion and extraction lumens can be varied, along with the types of infusion pumps or infusion pressure, etc, to ensure that the catheter can infuse and extract fluid at comparable flow rates. In some embodiments, referring to FIG. 3B, the cross sectional area of the extraction lumen 54 is larger than the cross sectional area of the infusion lumen 52. For example, the cross sectional area of the extraction lumen can be three times (3×) as large as the cross sectional area of the infusion lumen. Alternatively, the cross sectional area of the extraction lumen can be 2× as large as the cross sectional area of the infusion lumen. In addition to fluid infusion and removal, one or both lumens 52 and 54 can be sized for advancement of an advancement member, guidewire, endoscope or other viewing device, a sensing member, tissue biopsy device, or other minimally invasive surgical device.

As shown in FIG. 3A, infusion lumen 52 extends the entire length of the catheter. However, the infusion lumen does not carry solution 20 the entire length of the catheter. Rather, a bulkhead 53a occludes the infusion lumen slightly proximal to the middle of the catheter. A second bulkhead 53b is positioned distally to the first bulkhead 53a to form a pressure measurement chamber 55 in the lumen. In one embodiment shown, a pressure device 120c extends through the catheter, through bulkhead 53a, and into the pressure measurement chamber. In an alternative embodiment, the pressure device is located before the bulkhead 53a, enabling infusion flow to preserve fluid communication with the peritoneal cavity, but requiring cessation of the infusion pump for an accurate cavity pressure measurement. The pressure sensing device can be a pressure sensor embedded within the catheter or a fluid column within the catheter than connects to an external (out of patient) pressure sensor, for example. In the case of a fluid column, the pressure sensors 120b from FIG. 1 can be zeroed in air upon attachment to the unit 40. These pressure sensors are typically mounted at a height corresponding to the elevation of interest for pressure measurements. The pressure device 120c can further include a braid or coil for strength to prevent kinking.

An integral catheter pressure sensor does not need to be flushed by the user before use. Zeroing of the integral pressure sensor can be automatically done by the controller upon connection of the sensor or after a specific time delay. Alternatively, the controller may have an internal pressure sensor within the enclosure to measure ambient pressure, enabling the use of absolute pressure sensors in the catheter with no need of zeroing the pressure signal. Electrical connection of the integral pressure sensor may be by flex circuit or connection pads on the proximal end of the catheter. Calibration information for the integral sensors may be embedded in the connector of the catheter, in an RFID chip, in a bar code, or another method. The catheter may include one or more temperature sensors. Temperature information from the region where pressure is measured can be used to compensate for thermal drift in pressure sensor signal. Temperature measurement within the peritoneal cavity, preferably measured near the catheter infusion holes or within the infusion lumen, can be used as feedback to ensure that infusate temperature remains within desired/safe limits. Alternatively, calibration information for the integral pressure sensors may be on the catheter package and scanned by the controller during device set-up.

The pressure sensor 120b can be configured to detect the pressure in the peritoneal or other cavity, such as a bladder or other body cavity or organ, for example. In another embodiment, the pressure sensor can detect the pressure in the catheter and/or the patient cavity. This pressure signal can then be utilized by controller 41 in a feedback loop to automatically increase, decrease, or shut off infusion when the measured peritoneal cavity pressure varies from a selected absolute threshold or rate of increase. Desirably, the controller is configured to slow down the infusion rate as the selected pressure threshold is reached rather than shutting off infusion altogether. In use, such embodiments prevent or reduce the likelihood of over-pressurizing the peritoneal cavity. They also serve to optimize the rate of patient cooling (since the system need not be shut off to respond to over pressurization events) and allow the system to be run in a more automated fashion with less oversight by the user.

Also shown in FIG. 3A, the extraction lumen 54 is desirably continuous to the distal end of the infusion catheter. Additionally, the web 57 separating lumens 52 and 54 can be perforated distally to the second bulkhead 53b to increase the maximum extraction rate of the catheter. Alternatively, the web can be removed distally from the second bulkhead to form a single large extraction lumen.

An advancement member 30, such as a stylet, can be inserted into a lumen to provide column strength and prevent kinks during deployment of the catheter. The advancement member can be placed in the infusion lumen, the extraction lumen, or both, for example. In one embodiment, the inner diameter of the lumen can neck down near the distal end of the catheter so as to be able hold the advancement member by an interference fit. The advancement member can comprise any biocompatible material, as known in the art. In one embodiment, a stylet can include a tube that fits over the proximal end of the catheter. When the catheter is in place in a body cavity, the access device 60 can be removed by sliding over the catheter to engage with the stylet tube and pull the stylet out proximally from the catheter. In yet another embodiment, the access device can be slid proximally down the catheter and connected to the hub to provide strain relief to the catheter connection to the hub. The catheter can further include a floating coil in one of the lumens for column strength and to further prevent kinks and decrease friction between the catheter and stylet during stylet withdrawal. The pitch of the coil can be adjusted to tune the radial compliance of the catheter. The pitch of the coil may tight enough that the coils are edge to edge in regions along the catheter so the coil can acts as a infusate filter as well. Catheter reinforcement and kink resistance may also be accomplished through other means such as composite structures, braid reinforced walls or other means. Steerability features may also be included. Lubricious coatings may also be used to facilitate insertion and withdrawal of the advancement member.

In many cases, the advancement member will be sized for advancement in the extraction lumen or other lumen besides the infusion lumen so as to allow for the infusion of solution through the catheter during insertion. While in many cases it will be sized to be removably positioned in the catheter, in other embodiments it can also be fixed in position within the catheter.

The catheter can also include weights 59, which can be positioned at the distal tip of the catheter (or other locations). The web 57 at the distal end of the catheter can be removed or pushed aside, such as by melting, to allow the weights to fit in the entire diameter of the catheter. The weights can alternatively be placed in only one of the lumens, such as the extraction lumen or the infusion lumen, for example. The weights can comprise tungsten or other MRI compatible materials, or alternatively, can comprise stainless steel, or other non-MRI compatible materials. The weights are configured to cause the distal end of the catheter to “sink” to the bottom of the patient cavity to maximize the distance between the middle or proximal end of the catheter (where fluid is infused from) and the distal end of the catheter (where fluid is extracted from). The mass of the weights can be from 2 to 20 grams though other values are being contemplated. Weights may also be located along various locations over the length of the catheter.

Catheter 50 will also typically include one or more apertures 51 positioned along the infusion and extraction lumens to provide for the infusion and extraction of fluid from the peritoneal or other cavity. Typically, the outflow or infusion apertures 51a will be placed more proximally than inflow or extraction aperture 51c to reduce the pressure for infusion, and reduce the immediate uptake of the infused solution by the extraction apertures. In some embodiments, the infusion apertures more proximally positioned on the catheter may be smaller than the infusion apertures more distally positioned on the catheter to improve the even flow of fluid into the cavity. Similarly, proximal extraction holes may be smaller than distal extraction holes to make extraction flow more even across multiple holes. In one embodiment, the diameter of the apertures is smaller than the internal diameters of the infusion and outflow lumens of the catheter, or of the smallest internal diameter in the system, to prevent obstructions from entering the system. In one embodiment, the extraction apertures have a diameter of approximately 0.035″ to 0.045″ and are spaced approximately 0.2″ from each other. In another embodiment, the infusion apertures have a diameter of approximately 0.035″ to 0.045″ and are spaced approximately 0.25″ from each other. Although the apertures are shown in FIG. 3A as being in a row along a longitudinal axis of the catheter, in some embodiments the apertures can be positioned radially around the device. In some embodiments, multiple apertures are disposed along a transverse section of the catheter.

Pressure apertures 51b are placed within the pressure measurement chamber 55 to allow the pressure sensor 120b to be in fluidic communication with the patient cavity. Typically, there is a minimal amount of spacing between each aperture (e.g. 1 mm or greater) to improve flow rate and reduce clogging. The cross sectional area of the sum of apertures is typically significantly greater than the cross sectional area of the extraction lumen. In one embodiment, the cross sectional area of the sum of the apertures approximately 0.6 in² to 1.5 in². This can decrease the flow rate of fluid into the extraction lumen, which decreases the potential to pull body cavity contents and/or tissue against the catheter. Primary extraction suction occurs in the most proximal extraction holes. Spacing between the holes can be increased to provide drainage along a greater length of the catheter which correlates to more zones within the cavity being treated. One alternative design involves extracting fluid from the distal end of the catheter by only forming extraction holes in one lumen of the catheter with communication between the lumens only at the distal end of the catheter, near the weighted tip. In addition, the radial geometry of the catheter may be increased in the region of the extraction holes to hold cavity structures and organs away from the extraction holes. The radial geometry may be in the form of ridges, walls, columns, hairs, a helix, rings, T-shapes and the like. Ideally, the radial geometry can be groomed down for delivery through the access port and expand beyond the internal diameter of the access port once in the cavity. Another embodiment may use mesh, foam, or other porous material such as elastic porous tubes or filter material. Extraction port size may vary from 0.010″ to 0.1″, for example. Experiments using abdominal tissue have shown that smaller diameter holes are less susceptible to obstruction from either invagination or foreign bodies/particulate.

Several embodiments of the invention include a device for holding the catheter in place within a patient. One such device can be an adhesive pad that is adhered to the patient to hold the catheter in place. An example of this type of adhesive device is the StayFix device manufactured by Merit Medical Systems, Inc. In another embodiment the catheter can include a pre-formed bend at specific locations along the catheter to keep it in place after insertion into a patient cavity. For example, a 90° bend in the catheter at a predetermined position along the catheter could ensure the bend to occur within the patient cavity and provide additional resistance to accidental withdrawal of the catheter from the patient. A bend could optionally be pre-formed in the catheter at a position proximal to the insertion point of the catheter for the same effect. In another embodiment, a non-perforated balloon may also be located on the catheter within the cavity. When the balloon is inflated, it can act like an anchor to prevent catheter withdrawal. The internal balloon may have additional utility when coupled with external fixation of the catheter by applying compression and a seal to the tissue access tract to combat bleeding. The balloon may also be located on the catheter within the access channel creating pressure to control bleeding while simultaneously anchoring the device. Balloons on the outer diameter of the catheter may also be periodically inflated throughout treatment to push cavity tissues away from catheter extraction holes to ensure consistent extraction flow. In FIG. 8, balloons on either side of extraction holes can be inflated to put organs and other tissues away from the holes.

System 10 will typically include one or more sensors which can be selected to measure, flow rates, pressure, temperature, shivering, or other physical property. In some embodiments, the system will include at least one temperature sensor in the patient to measure patient temperature. The input from temperature sensor 120a can be utilized by controller 41 in a feedback loop to regulate infusate flow rate and the infusate temperature, so as to reduce the patient's temperature a selected amount as part of a hypothermic treatment regimen. Such placement options can include peritoneal, intravascular, auricular, oral, epidermal, esophageal, nasopharyngeal, bladder, tympanic, intramuscular, intra-abdominal wall, pectoral, and rectal/urethral. In some embodiments, peritoneal and/or intravascular temperature can be used for control purposes. However, in various embodiments, temperatures can be sampled from multiple locations and a composite temperature can be developed and used for control purposes, with assignable weightings to each location. In use, a composite measurement can give a more accurate reflection of the patient's temperature particular during fast cool regimens. In these embodiments, a temperature map can be developed and displayed to show the progress of cooling over the patient's body (e.g., as a wave or depth of cooling). In other embodiments, only temperature measurements from a particular target site to be cooled can be used, e.g., the peritoneal cavity. Temperature sensors can also be placed within the catheter to monitor the temperature of infused and extracted fluid. Shivering sensors can include accelerometers or pendulums to sense vibration, and strain gauges, lvdts, or string potentiometers to sense strain on the skin. Alternatively, shivering could be sensed with a pressure sensor placed within a muscle group. These non-invasive shivering sensors can be used by the controller in a feedback loop to regulate infusate flow rate and temperature for therapeutic benefit to the patient. In some cases, sensors may be combined into one peripheral device, such as an esophageal temperature probe that extends into the stomach for peritoneal cavity pressure measurements, or a intra-muscular temperature sensor that can also sense intra-muscular pressure for shivering detection. In some embodiments the temperature sensor for induction may be different that than the temperature sensor for maintenance due to differences in sensitivity, and patient comfort.

Catheter 50 can be fabricated from various biocompatible flexible polymers known in the art such as polyethylene (HDPE and LDPE), silicone, polyurethane, PTFE, Nylon, PEBAX and like materials. All or a portion of the catheter can include a lubricous coating such as a silicone coating or hydrophobic or hydrophilic coatings to assist in advancement of the catheter through tissue or the access device. Also, the tip or distal portions can be tapered. The catheter can also include braiding, coiling, or other means for improving kink resistance and increasing the burst strength of the catheter lumens. In particular embodiments, the proximal portions of the catheter can be braided or otherwise stiffened such that the catheter has sufficient column strength to be advanced into the peritoneal cavity through manipulation of the proximal portion of the catheter. In some embodiments, the catheter can include a handle (not shown) positioned at the proximal end of the catheter to assist in advancement of the catheter. The catheter may also include a perforated balloon to distribute fluid radially and serve as an anchor.

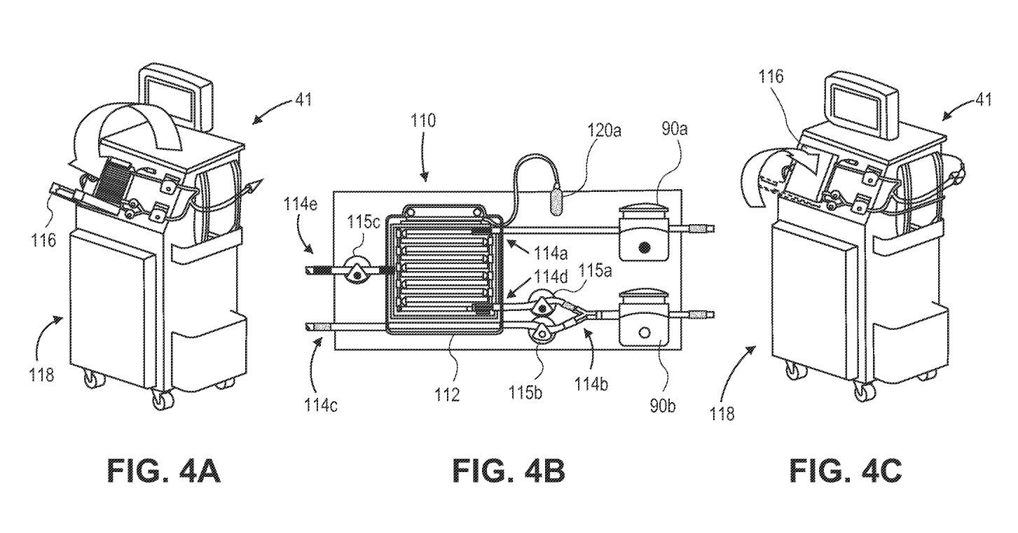

FIGS. 4A-4C further illustrate the heat exchanger assembly and how the assembly is fluidly connected to the rest of the hypothermia system. Heat exchanger assembly 110 can comprise various cooling and/or heating devices known in the art including electronic cooling devices, chillers, cryogenic gas based cooling devices, electric heaters, resistance heaters, heat sinks with forced ambient air, thermoelectric devices, and the like. In one embodiment illustrated in FIGS. 4A-4C, heat exchanger assembly 110 comprises a heat exchange surface (such as a thermoelectric surface) and a heat exchanger module 112 having fluid channels. The fluid channels can be heat formed tubes, for example. In FIG. 4B, the heat exchange surface is not illustrated because it is positioned under the heat exchange module 112. The heat exchanger module 112 can be removably placed and mated into firm contact with the thermoelectric device with door 116, for example. In some embodiments, the heat exchanger module can comprise aluminum or other similar materials. The thermally conductive surface in the heat exchanger may have fins or other features to provide greater thermal contact area with the infusate. Features like fins, screws, and epoxy mixing tips may also be added to mix the fluid to provide additional heat transfer. An electrically isolating but thermally-conductive material can be placed between the thermoelectric device and the heat exchanger module to provide electrical isolation between the thermoelectric and the heat exchanger module. Alternatively, the cold plate of a thermoelectric assembly can be made from a thermally-conducting, electrically isolating material such as aluminum nitride. The plate can be approximately 6″×6″ in size, for example. In another embodiment, the heat exchanger can use room temperature air (typically colder than body temperature) to cool the fluid outside of the patient. For example, the heat exchanger could include a fan to blow room temperature air across the infusate. Alternatively, the walls of the infusate tubing could be thin enough to allow room temperature air to cool the infusate therein. Another embodiment involves a fan that blows air on a heat sink that is in either direct or indirect thermal contact with the infusate. A refrigerator in the enclosure may also be used to cool infusate or house a cold reservoir of infusate used to fill and drain the patient during treatment.

In one embodiment, the heat exchanger assembly can comprise a compressor/evaporator circuit coupled to a boot plate. The compressor/evaporator circuit can be used to cool the infusate, and a separate resistive heater can be used to warm the infusate. The separate resistive heater can also be coupled to the boot plate, or alternatively, can be coupled to the heat exchange assembly. In another embodiment, a waste refrigerant system could be used to cool the evaporator/boot plate. Such a system could comprise a refrigerant source, inlet valve, metering orifice, and an optional outlet pressure regulator. The evaporator boot plate temperature can be monitored, and the inlet valve can be PWM modulated as necessary to vent appropriate amounts of refrigerant into the evaporator producing simple cycle refrigeration and maintaining the boot plate at a fixed temperature. An optional output pressure regulator could limit the resulting minimum temperature producible, if necessary. This embodiment would eliminate the need for a compressor and Peltier device, but would require that the refrigerant source tanks (e.g., Nitrous Oxide, Carbon Dioxide, 1,1,1,2-Tetrafluoroethane, or virtually any other refrigerant) be changed as needed. This system would reduce the electrical power requirements of the design significantly; making portability and operation on battery power more feasible, and making the design smaller. Additionally, the instantaneous cooling power of such a system could make it possible to use room temperature infusate and cool it in a more instantaneous “on demand” fashion.

In one embodiment, the heat exchanger can comprise a coil, switch-back, or other shape of aluminum or other metal tubing. This embodiment provides a simple, inexpensive part with lots of infusate contact. The boot plate may have a grooved surface that mates with the conductive tubing of the heat exchanger. In other embodiments, the heat exchanger tubing can be square in cross section for greater contact area with the boot plate.

Also shown in FIG. 4B, an infusion lumen of a catheter (not shown) can be coupled to the heat exchanger assembly 110 with tube 114a, and an extraction lumen of the catheter can be coupled to the heat exchanger assembly with tube 114b. Tubes 114a and 114b can also be referred to as patient lines. The tubes serve to provide a fluid communication between the heat exchanger module and the infusion and extraction lumens of the catheter. Referring still to FIG. 4B, it is shown that pumps 90a and 90b are configured to receive the tubes 114a and 114b. The pumps can be peristaltic pumps for example, and can be configured to infuse and extract fluid into and out from the patient without contacting or contaminating the fluid within the tubes. This feature is important because it allows the pump system to be reused for future patients with a new LAS 130. In some embodiments, the patient lines can include a reservoir to reduce/eliminate the pulsatility of fluid flow from the pumps. The reservoir can be, for example, a bag with flexible walls or a rigid container. In some embodiments of a rigid container reservoir, it can include an air volume within configured to contract and expand with pressure fluctuations. It should be noted that the infusion and extraction lumens may first pass through or be attached to other elements described above before reaching the heat exchanger assembly, such as LAS 130 and hub 140, as shown in FIG. 1. Tube 114b may be a Y-splitter that passes through a pair of pinch valves 115a and 115b to allow direction of waste or extracted fluid towards either waste fluid container 80 (via tube 114c), or back into the heat exchanger assembly for recirculation into the patient at tube 114d. Further details on recirculation will be discussed below. The heat exchanger assembly may draw fluid from fluid reservoir(s) 70 via tube 114e, which can also pass through a pinch valve 115c. The controller 41 can control the pinch valves to change the flow paths of fluid to and from both the fluid reservoirs and the waste container.

The fluid reservoirs may optionally be stored in refrigerator 118 to keep solution 20 at a desired temperature prior to infusion, as shown in FIG. 1A. In use, such embodiments allow for the immediate infusion of a hypothermic solution 20 without having to wait for a cool down period. In another embodiment, ice blocks may be used to cool the fluid and/or fluid reservoirs. The ice blocks can be changed out with new ice blocks as necessary to maintain the desired fluid temperature. The fluid reservoirs may be removable/disposable to allow for easy connection and disconnection to and from the system. The fluid reservoirs may also serve as waste reservoirs during and/or at the conclusion of treatment. The waste reservoir 80 may be a separate compartment integral to the fluid reservoirs 70, formed by sealing three layers of vinyl (or equivalent) together, rather than 2 layers. At beginning of treatment, fluid would reside in the fresh infusate side. Fluid from the patient could be pumped in and out of the waste side of the reservoir during treatment. An insulative layer may be used between the reservoirs to minimize heat transfer from waste fluid to the cold fluid side.

One or more temperature sensors can be placed in reservoir 70, container 80, as well as on heat exchanger assembly 110, so as to send input signals to controller 41, which can use these signals to optimize the cooling process using various control algorithms (e.g., PID, PI, etc) embedded within a thermal control module. These and related embodiments allow for rapid and continuous cooling of infused fluid 20 with reduced cooling power requirements. In various embodiments, the assembly can also include a supplemental cooling device to provide for faster cooling rates. The supplemental device can be Peltier thermoelectric or other cooling devices described herein.

In many embodiments, unit 40 can be integral or otherwise include one or more of reservoir(s) 70, waste container 80, pumps 90a and 90b, and heat exchanger assembly 110 so as to comprise main unit 40. In some embodiments, the main unit is a portable unit and will typically include a handle and is sized to be readily carried and transportable in an ambulance, EMT vehicle, crash cart and the like. The main unit can also include brackets or other mounting means to be quickly mounted to an IV pole, patient's bed, gurney, or like structure, or may also include an integral IV pole. The pole may be telescoping or otherwise self-expanding. The pole may allow specific components of unit 40, such as fluid reservoirs 70 and/or waste container 80, to be placed at various locations around the patient and also provides a gravitation head pressure for delivery of fluid 20 into or out of the patient. The pole can be raised or lowered to provide greater or lesser amounts of head pressure, which can be sensed via means of a pressure sensor placed in the catheter 50.

The reservoirs and waste containers can also include weight sensors, which can be used to measure the amount of solution in each of the reservoirs and/or waste container to determine the total volume of solution that has been infused into a patient. The weight sensors can also measure flow rate of solution in/out of the patient. In the case of a combined waste and infusate reservoir, a single weight sensor would be sufficient to control volume of fluid during treatment. The main unit 40 can include an integral battery, such as a rechargeable lead acid or nickel metal hydride battery having sufficient capacity for multiple hours of operations. The unit can also include electrical power connections for connection to an external power supply which can be either an AC or DC power supply.

Pumps 90a and 90b are desirably configured to provide sufficient pressure for fluid flow from reservoir 70 through catheter 50 and into cavity. In various embodiments, the pumps can comprise bi-directional pumps 90a and 90b, such as displacement pumps, peristaltic pumps, gear pumps, diaphragm pumps and the like. In some embodiments, the pumps are configured to infuse and extract fluid into a patient without contacting the fluid, to preserve the sterility of the fluid. The pumps can also be configured to interface with a pump cassette portion of the catheter such that the pumps do not need to contact fluid. The pumps can also be configured as a vacuum source by pumping in an opposite direction. The pumps or other pressure source, desirably provides sufficient pressure to infuse 2 to 6 liters of solution 20 into the peritoneal cavity of a patient in ten minutes or less. The pumps are desirably automated and can send and receive one or more inputs from controller 41. One alternative embodiment has only one infusion pump and uses the pressure in the patient cavity and gravity to drain the patient from the height of the bed to the waste reservoir. Another embodiment involves a peristaltic pump head on the extraction side of the system that is missing a roller. This feature enables the system to periodically release vacuum pressure in the extraction line, thus minimizing the potential for invagination obstruction of the catheter

In particular embodiments, a pump or pumps can be configured to produce pulsatile flow (for either infusion or removal of solution) and can include a selectable pressure and/or flow wave form such as sinusoidal, square wave and like waveforms. The period of the waveform can also be synchronized with one or more of heart rate, respiration as is described herein. In one embodiment, infusate flow can be counter-pulsated (e.g., approximately 180° out of phase) with the heart rate so as to increase blood flow through the peritoneal vasculature and provide a measure of pumping assistance to the heart. In related embodiments, such counter pulsation or other forms of synchronized flow can also be used to increase the patient's blood pressure (by producing vasoconstriction within the peritoneal and surrounding vasculature) for treatment of patients suffering blood loss, shock or other conditions causing low blood pressure. In various embodiments, the waveform and periods of infusion and removal, as well as synchronization, can be controlled by a duty cycle module executed by the controller 41. Synchronization can be achieved through inputs of one or more sensors, as well as inputs from external biomedical monitoring instrumentation. Flow pulsatility can increase peak fluid flow and concomitant suction at individual holes, increasing the potential for obstruction. In the event that flow pulsatility is not desirable, as in the case of extraction flow, a chamber can be added to the extraction patient line to diminish the pressure fluctuations and provide for more even extraction flow into the catheter. Alternatively, non-pulsatile pumps, such as gear pumps may be used. Extraction flow rate is related to reliability of flow with slower rates providing greater reliability. Extraction rates of 50 to 200 ml/min are typical.

Waste fluid can either be emptied into external waste container as illustrated in FIG. 1A, or into a waste container attached to a pole, or a waste container housed within the refrigerator. Preferably, waste container 80 and the connecting tubing are placed below the patient to provide for removal of fluid using gravitation head pressure alone (similar to reservoir 70, container 80 can be configured to be raised or lowered to vary the removal pressure). In such embodiments, the system can be configured so as to not require a pressure source, but instead completely rely on gravitation head pressure alone for both functions. Such embodiments provide an increased measure of portability for field use since no pressure or vacuum source is required. Particular embodiments of this configuration can be further adapted for battlefield or other emergency medicine use through the use of one or more of light weight weather resistant components, power efficient and fault tolerant processors and circuitry, rechargeable high volume efficiency batteries (e.g., lithium or lead acid), and even the use of light weight manual pumping devices. Another embodiment does not include a separate waste container 80, but rather returns waste fluid back into reservoir(s) 70 after infusion into the patient. In one embodiment, the system includes two reservoirs 70. The patient cavity can first be infused with solution from the first reservoir. Next, the fluid can be removed from the patient back into the first reservoir while additional cold solution is infused into the patient from the second reservoir. This scenario does not require an additional waste container. Still another embodiment involves a reservoir with a partition in the middle forming two chambers. At the beginning of treatment, fluid begins in one chamber and is delivered to the patient. When the patient is drained, fluid is withdrawn to the second chamber. This design does not require a separate waste reservoir and having a single volume measurement of fluid outside of the patient. The partition wall in the reservoir may have thermally insulating properties to minimize transfer of heat between drained fluid and fresh fluid.

In other embodiments, the pumps can be replaced with a compressed gas source, such as a compressed air source. The compressed gas source will typically include a control valve which can be an electronic valve operably coupled to controller 41. The control valve and controller 41 can also be configured to produce the forms of pulsatile and synchronized flow and related waveforms described above.

In some embodiments, the gas source is a compressed oxygen source which can be externally coupled oxygen source or an integral source coupled to unit 40. The compressed oxygen source is desirably configured to provide sufficient total pressure for fluid flow into the cavity. It is also desirably configured to have a sufficient oxygen partial pressure to oxygenate the infused solution so as to be able deliver sufficient oxygen to peritoneal or other tissue to help increase the blood oxygen saturation of a hypoxic patient. The system can also determine lactic acid or carbon dioxide content in the infusate and deduce whether or not sufficient oxygen is getting to the abdominal contents.

Referring again to FIG. 1A, the oxygen source can be coupled to an oxygenation element or device such as a bubble oxygenator or hollow fiber oxygenator. This oxygenation device can be positioned within reservoir 70, an oxygenation chamber fluidically coupled to reservoir 70, or within a lumen of infusion catheter 50. The flow of oxygen into solution 20 can be controlled through the use of one or more oxygen sensors positioned within reservoir 70, or infusion catheter 50. Controller 41 can receive one or more feedbacks from these sensors to regulate the oxygen saturation of solution 20 using an oxygen control module which uses one or more control algorithms, e.g., PID, etc. Also, multiple oxygen sensors can be externally placed along the length of infusion catheter 50 in order to measure oxygen partial pressures within different locations within the peritoneal or other cavity as well as a rate of oxygen uptake by peritoneal tissue. Such measured oxygen partial pressure can be utilized together with measured peritoneal pressures and temperatures to more precisely control the infusion and removal of solution from the peritoneal cavity for a hypothermic, resuscitative, dialysis or other medical treatment using an infused solution.

In various embodiments of methods of using the invention, system 10 can be used to cool or warm the patient's body temperature at different rates and different temperatures. Generally, though not necessarily, the patient's body temperature that is cooled is considered to be their core temperature. However, in some instances, system 10 can be configured to produce a more localized cooling effect or otherwise preferentially cool a particular targeted region of the body to a particular temperature (e.g., the peritoneal region), or even a particular organ (e.g., the heart), or an extremity (e.g., the leg) without necessarily bringing the patients core temperature to that level. This can be facilitated by placement of one or more sensors at the target tissue site to be cooled (e.g., in the peritoneal cavity, or a needle sensor inserted into the extremity) and by selecting/creating an appropriate anatomical space for infusate to reside.

System 10 can be used to produce a particular hypothermic or cooling regimen (e.g. rate of cooling and target temperature). The cooling regimen can be titrated to treat a variety of medical conditions including stroke, myocardial infarction, blood loss or any condition causing reduced perfusion to the brain, heart or any of the major organ systems, e.g. the kidneys, gastro-intestinal, system, etc., as well as any extremity, e.g. arm, leg, etc. The system can also be used to produce hypothermic cooling in patients receiving CPR, which can offer improvements in circulation to the head and thorax. This can have similar benefits to “abdominal binding” where elevated pressure in the abdominal region directs more blood flow to the head and thorax. In particular embodiments, the cooling regimens can be employed to treat particular conditions e.g., stroke vs. myocardial infarction so as to reduce the amount of ischemic reperfusion injury to vital organs resulting from the particular ischemic event. In specific embodiments, the cooling regimen can be configured to do one or more of the following: i) reduce coronary infarct size and related sequelae from various cardiac events such as acute myocardial infarction, cardiac arrest, arrhythmia, trauma or other cardiac insufficiency; ii) reduce cerebral infarct size and related sequelia from stroke, cerebral vessel dissection, head trauma, cardiac arrest, arrhythmia, blood loss or other cardio-pulmonary insufficiency; iii) reduce tissue injury in other vital organs from cardiac arrest, blood loss or other cardio-pulmonary insufficiency; iv) reduce tissue injury in an extremity (e.g., the leg) resulting from trauma or blood loss; v) reduce post surgical tissue inflammation; and vi) provide a tissue protective effect from reduced perfusion resulting from surgery or other medical procedure.

In various embodiments, the cooling regimen can be selected by the user from a database of cooling regimens stored in memory resources within the main unit or an external device or computer wirelessly interfaced to system 10. The database of cooling regimens can include regimens for particular conditions, e.g., myocardial infarction as described above. The user can select a regimen from the database and use it unmodified or may customize, or otherwise fine-tune it to the particular patient and his/her current condition. This can be done by adjusting one more treatment parameters such as infusion rate, infusate temperature, target temperature, treatment duration, warming rate, etc.

System 10 can be configured to cool or warm the temperature to a variety of ranges. In many embodiments, the system can be used to cool the patient's temperature in the range of about 30 to about 35° C., with a preferred target temperature of 32.5° C. Lower ranges can also be selected depending upon the medical condition or surgical procedure. In embodiments for treatment of acute myocardial infarction or stroke, system 10 can be configured to cool the patient's temperature the targeted value (e.g., 34° C.) in ten minutes or less. In many embodiments, this can be achieved by rapidly infusing a bolus of chilled solution into the peritoneal cavity between approximately 2 to 6 liters. However, the volume of infused solution is typically optimized for each individual patient. Cooling times as short as five minutes to 34° C. or less are also contemplated and can be achieved through use cooler infusion solutions including solutions cooled below 0° C. Faster cooling can be achieved by infusing cooler solution and/or high infusion rates. Higher flow rates can be achieved through the use of higher pressure or larger lumen diameters for the infusion catheter 50. In particular embodiments, the lumen diameter of the infusion catheter can be configured for delivering a maximum flow rate and the medical care provider can select the infusion catheter for its maximum flow rate so as to deliver a desired amount of solution 20 for a particular medical condition.

Also in various embodiments, system 10 can be used to cool all or a selected portion of the patient's body prior and post surgery to reduce a patient's inflammatory response resulting from the surgery due to the release of cytokines etc. In related embodiments, system 10 can be used for pre-operative and intra-operative cooling of a selected operative site, such as the heart, to allow for extended periods of operation on the organ with reduced or no perfusion through the organ. In one embodiment, the system can be configured to chill the heart (similar to cardioplegia) to allow for various forms of cardiac surgery, which may require the heart to be stopped or where portions of the heart are cross clamped, such as valve replacement, CABG, aorta repair, atrial-septal defect repair and like procedures. This can be achieved through cooling of the peritoneal cavity, or by direct infusion of cooling solution to a chamber of the heart using a cardiac type port access device known in the art.

In such embodiments, system 10 can be configured to achieve coronary tissue temperatures in the range of about 20 to 25° C. or an even lower range for example 10 to 20° C. Lower temperatures can be selected and titrated for longer periods of cardiac arrest or reduced coronary perfusion. For example, for periods of cross clamping less than 60 minutes, a 20 to 25° C. range can be selected, while for periods in excess of 60 minutes a 10 to 20° C. range can be selected. Also, the system can be used to a provide a pre-operative period of hypothermic treatment, also known as pre-ischemic conditioning, to extend operating time and reduce an amount of post operative cardiac reperfusion injury.

In some embodiments, it may be desirable to gradually cool the patient. According to the literature, application of fluids at a low temperature can induce a shivering response and result in patient discomfort. To prevent this from happening, the system can expose the body cavity to small amounts of solution at a temperature at or near the normal patient temperature, then gradually lower the temperature of the solution added to the cavity at a rate that allows for sensory adaptation to the stimulus created by the chilled solution. In one embodiment, the solution will be introduced at a temperature of 37° C. Next, fluid can be added at a slightly lower temperature, such as 36° C. As the patient adapts to the chilled fluid, additional solution can be added at even colder temperatures. The solution can then be recirculated in the patient cavity to maintain a constant volume or pressure while also gradually reducing the temperature of the solution in the cavity. In another embodiment, the fluid may be introduced and allowed to dwell undisturbed for a period of time or until a certain change in core temperature, infusate temperature (Example: temperature delta, rate of change, or absolute value) to allow a temperature exchange between the patient and the infusate. When the desired treatment is completed, the patient can be warmed back to normal body temperature. During each of these steps, inputs from pressure sensors, temperatures sensors, etc can be used to manage the system 10, including heat exchanger assembly 110, pumps 90a and 90b, etc.

In other embodiments, the system 10 can include a device that can diagnose peritoneal hypertension by looking at the respiratory signal amplitude in the peritoneal pressure signal. The system can also use the presence of a respiratory signal to confirm that the catheter is connected to the LAS/controller and there is a fluid connection between the sensor and the patient (The pressure line has been flushed in the case of external pressure sensors).

Some exemplary modes of operation of system 10 will now be discussed. The system 10 optimally includes modes of operation that will be referred to herein as fill, irrigate, irrigate/drain, pre-drain, overshoot-stop, recirculate, transport, warming, drain, and auto-prime modes of operation, however other modes of operation fall within the scope of system 10.

A fill mode of operation can be an automated mode that fills a patient cavity with solution 20 through catheter 50 to a safe internal cavity pressure. In some embodiments, a safe internal cavity pressure may be from approximately 5-35 mm Hg, however other internal cavity pressures may also be deemed safe. Controller 41 of system 10 can utilize input from pressure sensors, such as pressure sensor 120b located in catheter 50, to form a feedback loop that monitors the pressure of the patient cavity. The flow rate of solution into the patient cavity can then be automatically adjusted by the controller (by adjusting pumps 90a and 90b) based on the sensed pressure. Typical flow rates during Fill mode may be 1 to 4 liters per minute, depending on the internal diameter of the catheter and the permissible pressure in the tubing set. In one embodiment, the infusion rate of a catheter during fill mode is approximately 1.3 to 2 liters per minute.

In some embodiments, patients can tolerate elevated peritoneal pressures for varying amounts of time. Generally, the higher the peritoneal pressure, the less time the patient can be tolerated. In some embodiments, the system targets a pressure of 14 mm Hg in the peritoneal cavity, a pressure that can be tolerated in patients ad infinitum. In another embodiment, the patient can be filled initially to a higher pressure, such as 25 mm Hg. This would provide more cold fluid in the patient to rapidly drop their core temperature. Immediately after filling the patient, the device can begin removing fluid from the patient so that the peritoneal cavity pressure would not remain elevated beyond the tolerable limit (e.g., 5 min at 25 mm Hg). In one embodiment, both the infusion lumen 52 and extraction lumen 54 of the catheter can be used to infuse solution into the patient cavity. Since each lumen is controlled by a separate pump head, the flow rate through each lumen may be independently controlled as a function of allowable pressure or flow rate. This mode of operation can also use other sensors, as described above, to control other elements of the system, such as using temperature sensors to control refrigerator 118 and/or heat exchanger assembly 110 to adjust the temperature of the solution. Typically in this mode of operation, the catheter can infuse solution into the patient from reservoir(s) 70, but does not remove any solution from the patient to waste container 80. Referring to FIGS. 4A-4C, during fill mode, pinch valve 115c can be opened to allow fluid to flow from reservoirs 70 through pump 90a into the patient, but pinch valves 115b and 115c can be closed to prevent fluid from flowing back through the heat exchanger assembly or into the waste container. An end point of fill mode can be, for example, when the cavity pressure in the patient reaches a threshold, such as 14 mm Hg, or when a preferred volume of fluid is infused into the patient, such as 6 liters total, or 2 liters for every <²° C. of desired core temperature drop, or 3 liters for every 2 to ³° C. of core temperature drop.

In some embodiments, the initial peritoneal pressure of a patient may be higher than the target “safe” peritoneal pressure during treatment, but the patient can be filled with infusate despite the initial elevated pressure. For example, if the target peritoneal pressure during treatment is 14 mm HG, and the patient's peritoneal pressure prior to treatment is higher than 14 mm HG, a minimum fill volume can still be infused into the patient. It has been shown that a minimum fill volume of 2.5 liters is a safe volume regardless of initial peritoneal cavity pressure.

In some embodiments, the fill volume can be adjusted based on the amount of cooling energy needed. If, for example, it is desired to cool a patient only a few degrees below normal body temperature, then smaller volumes of fluid can be infused into the patient. In one embodiment, the volume of fluid can be a function of initial patient temperature and/or patient mass entered by the user. Another method that could be used separately or in concert with changes in volume, would be to infuse the patient with fluid that is warmer than the typical 0° C. fluid. This can be done by using room temperature fluid, or warming the 0° C. fluid stored in the machine as it passes through the heat exchanger of the tubing set. Warmer fluid decreases the delta-T between infusate and patient, thus creating less cooling effect while still providing sufficient fluid volume for reliable flow in the patient.

In one embodiment, the patient is filled with body-temperature fluid. Then, the fluid can be cooled as it flows through the system and patient, thus cooling the patient. As the target temperature of the patient decreases, the system can cool the patient at any rate desired by the user. Rates could vary from 0.01° C./hr to 20° C./hr.

The irrigate mode of operation can also be an automated mode of operation. When the system 10 is in irrigate mode, catheter 50 can simultaneously infuse solution 20 into the patient from reservoirs 70 and remove solution from the cavity into waste container 80. Referring to FIG. 4A-4C, pinch valves 115c and 115b can be open, and pinch valve 115a can be closed, to allow fluid to flow from reservoirs 70 through pump 90a into the patient cavity, and back through pump 90b into waste container 80. The irrigate mode may automatically terminate once the patient is at a target temperature, or once the fluid reservoirs are empty. In one embodiment, the waste container 80 can also be stored in a refrigerator with the fluid reservoirs. If the reservoirs empty during irrigation, the fluid from the waste containers can be pumped into the fluid reservoirs to continue the irrigate mode of operation. In one embodiment, fluid drains from the patient under gravity drain (siphon) and is replaced with cold fluid in the patient based on feedback to the controller about the volume drained, cavity pressure, time, or other parameters.

Typically, the irrigate mode of operation maintains the pressure of fluid in the cavity that is measured at the start of the irrigate mode. The irrigate mode can also operate based on the volume of fluid infused and removed to the patient. In this embodiment, the irrigate mode of operation can maintain a constant volume of fluid inside the patient. This can be accomplished, for example, by weighing the volume of fluid in the fluid and waste reservoirs to determine the volume of fluid in the patient, and monitoring the volumes of fluid in both the waste containers and fluid reservoirs during irrigate mode to maintain the same volume of fluid in the patient. This can be advantageous if the pressure measurements from pressure sensors 120b are inaccurate or unavailable. An end point of the irrigate mode can be, for example, when the core temperature of the patient reaches approximately 34° C., or when the fluid reservoirs are empty.

An irrigate/drain mode of operation can also be an automated mode, and is similar in function to the irrigate mode. When the system 10 is in irrigate/drain mode, catheter 50 can simultaneously infuse solution 20 into the patient from reservoirs 70 and remove solution from the cavity into waste container 80. However, in contrast to irrigate mode, irrigate/drain mode drains the total volume of infusate in a patient to a preferred or predetermined volume, such as 2 liters of total infusate in the patient, for example. Referring to FIGS. 4A-4C, pinch valves 115c and 115b can be open, and pinch valve 115a can be closed, to allow fluid to flow from reservoirs 70 through pump 90a into the patient cavity, and back through pump 90b into waste container 80. In order for the system to drain the patient, the system must be pumping more fluid out of the patient than into the patient. Thus, an extraction rate of fluid from the patient must be larger than an infusion rate of fluid into the patient. The irrigate/drain mode may automatically terminate once the patient is at a target temperature, or once the fluid reservoirs are empty. In one embodiment, the waste container 80 can also be stored in a refrigerator with the fluid reservoirs. If the reservoirs empty during irrigation, the fluid from the waste containers can be pumped into the fluid reservoirs to continue the irrigate mode of operation.

In a related mode of operation, a pre-drain mode of operation can remove fluid from a patient to a desired treatment volume of infusate. For example, if the desired treatment volume is 2 liters, the pre-drain mode of operation can pump fluid out of the patient to achieve that target treatment volume. Alternatively, the end condition of pre-drain mode may be a treatment volume that is a percentage of initial fill volume. Referring to FIGS. 4A-4C, pinch valves 115c and 115a can be closed, and pinch valve 115b can be open, to allow fluid to flow from the patient cavity, and back through pump 90b into waste container 80. The pre-drain mode can terminate when the volume of fluid in the patient is equal to the target treatment volume. Pre-drain mode may be periodically interrupted to circulate fluid in a closed loop for assessment of fluid extraction flow based on infusion line pressure, infusate temperature, or other sensor inputs. Pre-drain mode may also be accomplished by allowing the patient to drain under gravity to a desired volume of fluid within the cavity.

The purpose of overshoot-stop mode is to minimize the patient temperature from overshooting the target temperature during rapid cooling. Overshoot stop mode may be initiated prior to reaching the target temperature or when the patient reaches the target temperature. An overshoot-stop mode of operation can be used to warm the fluid within a patient when the patient's core temperature reaches a predetermined temperature. For example, in one embodiment, overshoot-stop can be used when a patient temperature reaches 32.5° C. to arrest the patient cooling. Overshoot-stop mode may also use the infusate temperature as a end point when it reaches a typical temperature for hypothermia maintenance, such as 25° C.

A recirculate mode of operation can also be an automated mode, and is typically carried out after a fill or irrigate mode. When the system 10 is in recirculation mode, fluid can be infused into the patient at an infusion rate and extracted from the patient at an extraction rate. In some embodiments, the extraction rate is equal to the infusion rate. The fluid can then be recirculated through the heat exchanger assembly and cooled, and then pumped or infused back into the patient cavity. Referring to FIG. 4A, the recirculate mode can be accomplished by closing pinch valves 115c and 115b, and opening pinch valve. Fluid can flow through pump 90a into the patient, then be extracted from the patient with pump 90b, flow through the heat exchanger assembly, and back into the patient through pump 90a. As described above, sensors in the system can continually monitor the temperature, pressure, valve position, and tube installation in pinch valves etc, to ensure safe operating levels. Different recirculation times may be required depending on the type of injury sustained by the patient or the surgical procedure performed on the patient. For example, a cardiac patient may be on a recirculation mode for approximately 6-24 hours, while a spinal cord or brain injury patient may need to be on recirculation mode for up to 7 days. Patient temperature is maintained by modulating the thermoelectric power, pump speed, or a combination of both. This mode may operate with a indwelling large volume of fluid in the patient, or a minimal volume of fluid in the patient. Another embodiment involves permitting fluids within the abdomen to passively drain to a waste reservoir 80 by gravity/siphon/cavity pressure during recirculate mode. Additional fluid can be added to the patient in response to changes in the patient core temperature or other patient conditions.

In one embodiment, the recirculate mode can use ambient temperature to cool the fluid. Typically, infusate temperatures during recirculate mode are in the range of 20 to 25° C. Since ambient room temperatures in hospitals are typically lower than body temperature, allowing the patient tubing to be exposed to ambient air temperature may still provide enough cooling power to the system to maintain hypothermic temperatures in a patient once it's already been achieved.

A transport mode may be needed after a fill or irrigation mode in the event that a patient must be moved. The transport mode requires that the system be portable. As described above, unit 40 can be detached from certain system components, such as reservoirs 70, waste container 80, and refrigerator 118. When solution has been infused into a patient and the patient must be moved, a transport mode will allow recirculation to occur after the unit 40 has been detached from the rest of the system. A battery, for example, can be used to power the heat exchanger assembly and pumps, and fluid within the patient can be recirculated through the heat exchanger assembly to maintain a target temperature. In another embodiment, the pumps and heat exchanger assembly can be powered down during transport, and the battery can be used only to maintain power to the controller and user interface.

A warming mode of operation can warm a patient back to a desired temperature. In this mode of operation, instead of infusing a chilled liquid into the patient, as described above, the heat exchanger assembly or an optional heating device can be configured to warm the infusate. In another embodiment, the “warmed” solution is not necessarily a heated solution, but rather is just a fluid with a temperature higher than the previously infused chilled solution. The “warmed” solution may be, for example, a room temperature or body temperature solution. In some cases, and depending on the condition of the patient, the patient metabolism may warm the patient to an extent that the device is constantly cooling the patient during “Warming” mode. This warmed solution can be infused into the patient, as described above. The warming and recirculation modes of operation can work together to change a patient temperature depending on the patient's condition. For example, when a patient attached to system 10 operating in a recirculation mode reaches a stable condition, the patient can be re-warmed with the warming mode. If that patient's condition were to degrade (e.g., if a brain injury patients brain pressure reached dangerous levels) then the system could revert back to a cooling recirculation mode to stabilize the patient. One method of controlling warming mode is by incrementing the target temperature gradually in accordance with the desired warming rate.

Cooling and warming modes can also be performed automatically based on a sensed parameter. In many embodiments, a core temperature of the patient is monitored and the cooling and warming modes of operation are automatically used to control the core temperature of the patient. The core temperature can be monitored with, for example, an esophageal temperature sensor, a tympanic membrane sensor, and other temperature sensors on or inside the body previously described herein. In one embodiment, the cerebral spinal fluid pressure of a patient can be monitored. If the patient is in a cooled or hypothermic state, the system can automatically induce a warming mode of operation. If the system detects any spikes in the cerebral spinal fluid pressure, the system can automatically enter a cooling mode (such as fill, irrigate, recirculate, etc) to cool the patient. The system can also automatically attempt to rewarm the patient at predetermined intervals, such as every 4 hours, 8 hours, etc.

The system 10 can also operate in a drainage mode. In this mode of operation, the solution can be extracted from the patient cavity with extraction lumen 54 of the catheter into the waste container. Referring to FIG. 4, pinch valves 115c and 115a can be closed, and pinch valve 115b can be opened. Pump 90b can be used to extract fluid from the patient, such as through the extraction lumen of the catheter, and into the waste container. In another embodiment, as previously described, the system can drain the patient without pumps but instead by using gravity. Inputs from weight sensors in the waste container can be compared to the weight of solution infused in the patient to determine the total volume of solution still remaining in the patient. In one embodiment, when fluid stops flowing from the patient in the drainage mode, the patient is empty or nearly empty. In another embodiment, both the infusion and extraction lumens can drain fluid from the patient. It should be understood, however, that it is not necessary to remove all solution from the patient since any remaining solution will be absorbed by the body. In some embodiments, the drain mode can be used immediately after the fill mode. In another embodiment, there may be a pause between the fill and drain modes. The pause duration may be a function of time, a function of infusate temperature within the cavity, a function of the temperature of infusate sampled/extracted from the cavity.

In yet another embodiment, the system can operate in a mode that does not involve fluid flow into or out of the patient. In this embodiment, the system can pause fluid flow when the core temperature of the patient is in an acceptable range around the target temperature. Once the patient temperature has exited the acceptable range (either higher or lower), the system can respond with the flow of warm or cold fluid as needed. This mode can occur in a patient that either has a dwelling volume of fluid in a cavity, or an empty cavity.

In an auto-prime mode, fluid is flushed through the LAS, recirculation cap, and patient lines prior to connection with the catheter to remove air from the LAS and fill the patient lines with fluid from the reservoir(s). The recirculation cap at the catheter hub shunts fluid flow from the infusion lines to the extraction lines within the hub. The fluid that is flushed through the LAS can be diverted into the waste container 80. This sequence can be automatically stopped when fluid is detected in the waste container. After the auto-prime mode, the system can automatically enter a recirculate mode to cool down the solution and the heat exchanger assembly. In one embodiment, the pumps can run a short period after the fluid is flushed through the LAS to pull a vacuum on the fluid. This can prevent fluid from being lost from the lines when the recirculation cap is removed from the LAS after auto-prime. During auto-prime mode, the system can periodically pressurize the patient lines to detect conditions such as cap presence, catheter presence, or ambient pressure conditions using a leak down test, flow test, or other method.

In one embodiment, the system can include air sensors configured to detect air in the patient lines or catheter. In some embodiments, the air sensor can be mounted adjacent to one of the pump heads or valves so that installation of the tubing set into the pump heads or valves automatically locates the tubing into the bubble sensor. In some embodiments, the air sensors can be ultrasonic, optical, or conductive sensors. In some embodiments, detection of air or bubbles in the patient lines or catheter can result in the system responding by draining for a set amount of time or irrigating the patient in order to maintain volume of fluid in the patient. The air sensors can provide an input to the controller when there is air detected in the system. The system can redirect flow until it does not see a bubble for a certain amount of time (e.g., 3 sec or any other predetermined time period), depending on pump speed. The pump speed can be increased continuously or periodically to carry standing bubbles near the sensor on to the waste reservoir.

In another embodiment, the system can include passive air bubble management. In one embodiment, the system may have a reservoir in the fluid circuit that enables air bubbles to float to the top of the reservoir, rather than flowing further through the fluid circuit. The diameter of the reservoir can be sufficient that bubbles can float up to the top of the reservoir despite the flow rate of infusate during treatment. The reservoir can be located on the low pressure side of the system and vented to atmosphere. In other embodiments, the reservoir can be part of the heat exchanger. The reservoir could be on the high pressure side and vented to the waste bag by passive means (hydrophobic perforated material), or actively removed by sensing the air in the reservoir with a sensor and opening a valve that enables the air to flow to the waste bag or vent to atmospheric. The sensor can be ultrasonic, conductive, optical, resistive, infrared, or other types. The waste bag may be equipped with an air vent at the top to prevent over-inflation of the waste bag in the event that the system removes copious amount of air from the lines.

The vent may be a perforated hydrophobic material to prevent the loss of fluid.

A decrease in infusion line pressure in a recirculate mode can indicate the presence of either air in the infusion line or an extraction flow obstruction. The system can diagnose which condition exists by converting to irrigate mode. If the infusion line pressure immediately goes up to a normal level (e.g., 3 to 5 psi), the infusion line is likely filled with fluid (no air) and there is an extraction flow problem. If the infusion line pressure does not immediately go up, the infusion line is likely filled with air.

The system 10 can also automatically detect problems, such as obstructions or leaks in the fluid lines or in the catheter. In one method, system can detect an obstruction when the infusate temperature deviates beyond a characteristic amount. For example, to achieve therapeutic hypothermia, it may be desirable to have an infusate temperature 8° C. less than the core temperature of the patient. If the infusate temperature starts to rise (i.e., the infusate temperature approaches the core temperature of the patient or ambient temperature) then it can be an indicator that there is an obstruction or blockage within the system. The catheter can then be removed to find and eliminate the blockage. Another method to detect flow obstruction during open-fluid path treatment (for example, Irrigate mode) involves monitoring the rate of fluid accumulation in the waste bag. When fluid removal drops below a certain rate (typically 100 ml over 30 seconds), the system may enable an obstruction prevention routine or simply add fluid to the patient. In a closed-fluid path more (for example, Recirculate mode), the Infusion line pressure can be used to detect fluid flow. In the event of a catheter obstruction, flow does not enter the catheter. It follows that fluid flow through the tubing and out the infusion line would be zero with concomitant low or zero infusion line pressure. Another method to detect catheter obstruction is to monitor the fluid pressure within the infusion Patient Line. In a closed loop mode, such as Recirculate or Warm mode, a drop in infusion line pressure indicates that fluid is not entering the system from the catheter.

The system 10 can alter the fluid flow path and flow rate as a periodic prophylactic procedure and/or in response to a sensed obstruction. The direction of the pumps can be reversed to dislodge the obstruction(s), or the system can be re-primed in response to a detected obstruction. In another embodiment, the system can reverse the direction of the pumps periodically at a pre-determined interval (e.g., every 10 minutes the direction of the pumps can be reversed for 30 seconds) to periodically dislodge any obstructions within the system. In yet another embodiment, the system can detect a leak in the fluid lines or catheter when the cavity pressure drops. In yet another embodiment, the system can push a nominal amount of infusate (200 ml for example) from the infusate bags through the extraction lumen to push an obstruction out and away from the catheter. The system then removes the same volume of fluid through the extraction lumen and may returns it to either the infusate bags or the waste bag. Alternatively, the system may not remove the entire amount of fluid infused through the extraction lumen in order to maintain the treatment volume of the patient in response to infusate absorption out of the treatment cavity.

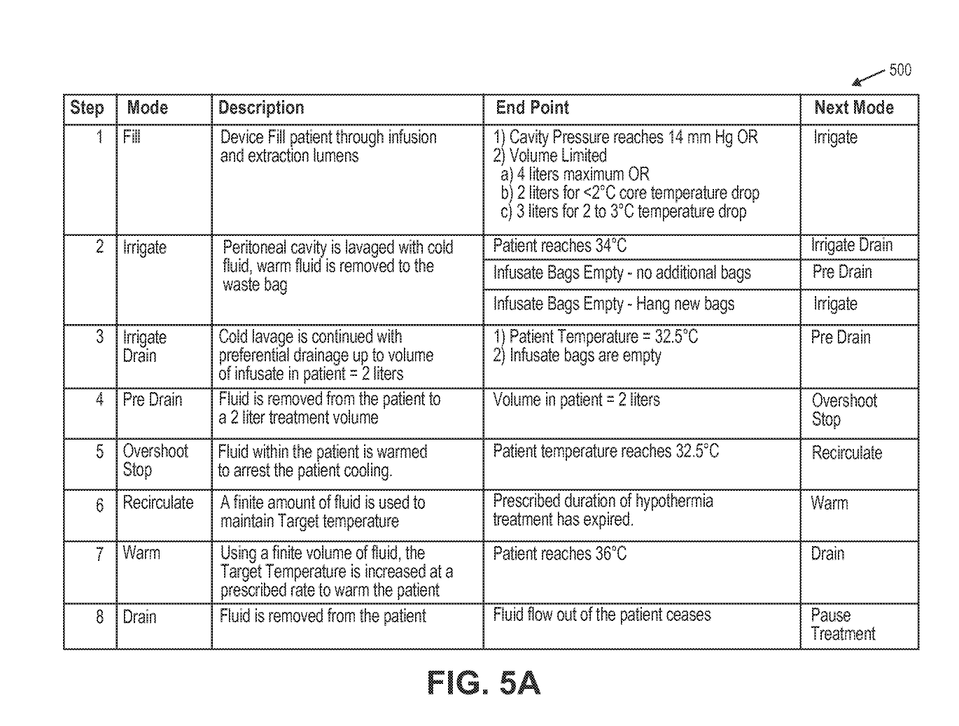

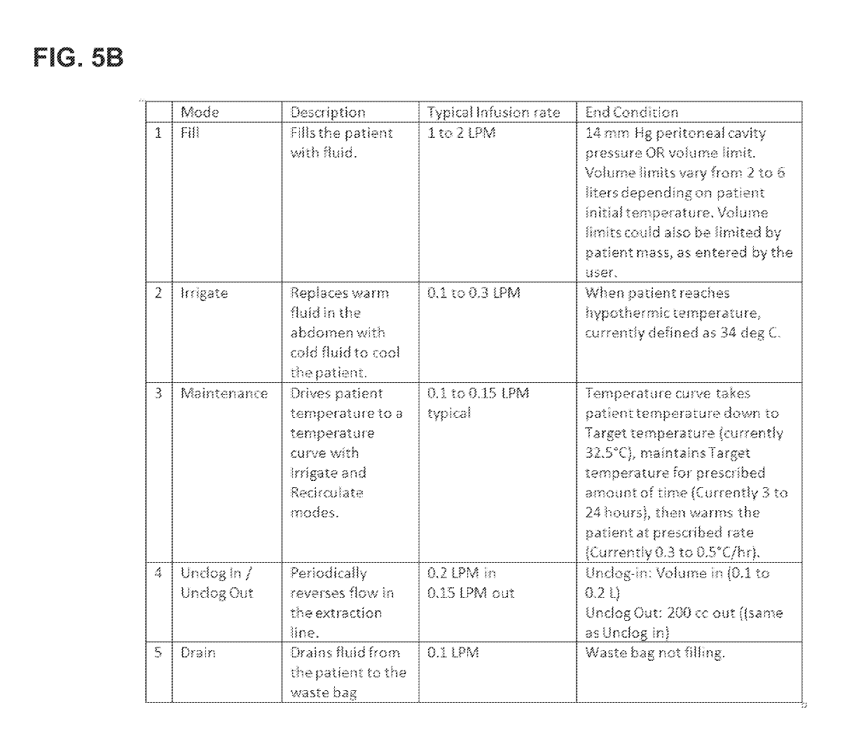

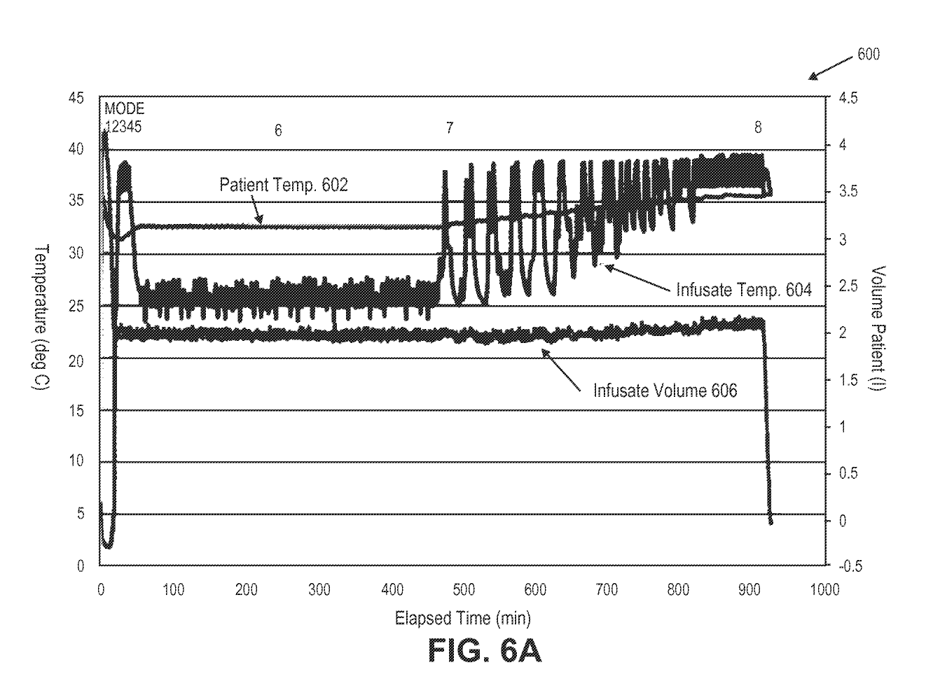

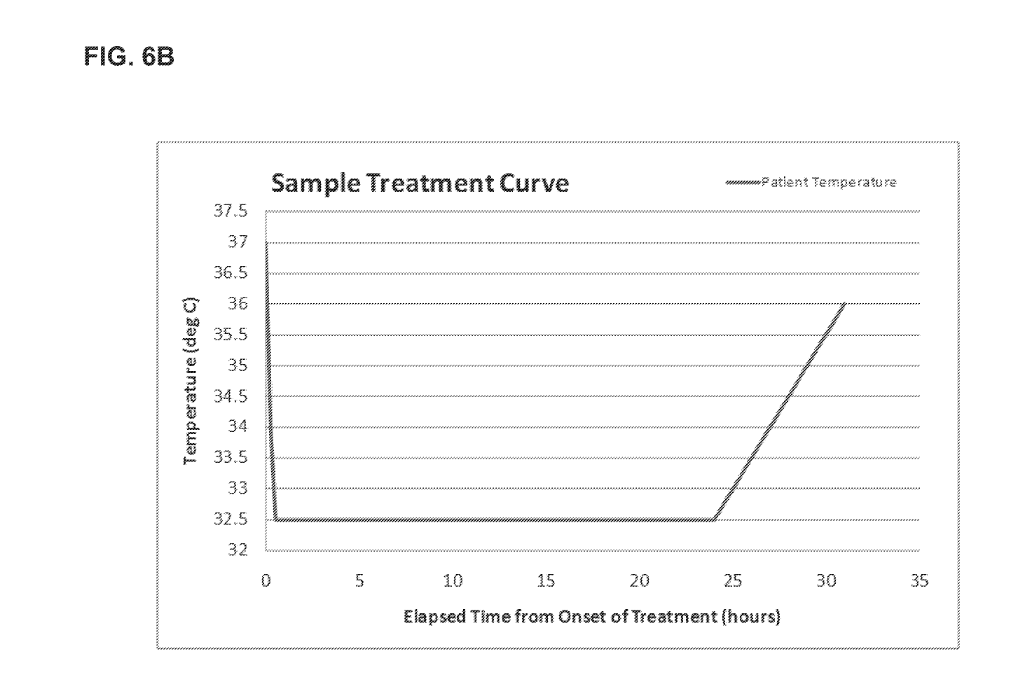

Referring now to FIGS. 5A and 6A, one example of patient treatment will now be described with respect to the modes of operation discussed above. Flowchart 500 of FIG. 5 represents one embodiment of the various modes of operation that can be used during treatment of a patient. Plot 600 of FIG. 6 illustrates the patient temperature 602, infusate temperature 604, and infusate volume in the patient 606 during the patient treatment.

At step 1 of flowchart 500, the fill mode of operation is used to fill the patient with fluid through the infusion catheter. It can be seen in plot 600 of FIG. 6 that the patient temperature 602 starts at 37° C. and begins to drop as the fill mode is initiated (see patient temperature 602 corresponding to mode 1 on plot 600). The infusate temperature 604 remains very cold (approximately 2-4° C. in FIG. 6) and the infusate volume goes up (to a maximum of about 4-6 liters). FIG. 6 shows the maximum infusate volume being around 4 liters.

In some embodiments, the patient may present with a core temperature lower than normal. In these instances, the fill volume can be limited to a smaller volume to mitigate against the potential for the system to overshoot the target temperature. For example, if a patient presents at 34° C. prior to treatment, the fill volume may be lowered (e.g., 2 liters instead of 4-6 liters) so as to cool less rapidly towards the target temperature (e.g. 32.5° C.).

When the end point of the fill mode is reached, as described above, the system can go into irrigate mode at step 2 of flowchart 500 to decrease the temperature of the patient towards a first set point temperature. In the irrigate mode, the body cavity of the patient can be lavaged with a cold fluid, and fluid within the cavity can be removed to a waste bag of the system. The irrigate mode can provide maximal cooling to the patient. Reaching the first set point temperature rapidly is important so that the patient reaches a therapeutic temperature as quickly as possible. As seen in FIG. 6, the patient temperature 602 continues to drop and the infusate temperature 604 rises. The infusate volume 606 can remain constant during irrigate mode. In some embodiments, a patient may require additional cooling energy to rapidly cool to hypothermic temperatures. In one embodiment, the controller may assess the core temperature of a patient after a pre-determined time period (e.g., after 5 minutes of irrigate) and make adjustments to the treatment algorithm if the patient is not responding to the cooling therapy as desired. For example, the system may add additional fluid to the patient within an acceptable pressure range, or decrease the first target temperature of the algorithm to ensure a longer period of irrigate mode resulting in faster or more effective cooling.

If the patient reaches the target temperature (e.g., a first set point of 34° C. in some embodiments) then the system can go into irrigate/drain mode at step 3, recirculate mode at step 6, or remain in irrigate mode. If the fluid reservoirs are emptied and there is no additional infusate, the system can go into the pre-drain mode at step 4. If the fluid reservoirs are emptied but there is additional infusate available to the system (e.g., extra infusate bags) then the system can remain in irrigate mode. Upon reaching the first set point temperature, a cooling algorithm in the controller can automatically select the next mode of operation based on the current patient core temperature, the current patient cooling slope (e.g., the rate of cooling or rate of core temperature drop), and/or the expected cooling slope.

If the system enters irrigate/drain mode from the irrigate mode, at step 3 the cold lavage of fluid into the patient is continued with preferential drainage until the volume of infusate in the patient is approximately 2 liters. In other embodiments, the preferred volume of infusate can be lower or higher than 2 liters. During the irrigate/drain mode, the infusate volume 606 can be lowered to approximately 2 liters, the patient temperature 602 can approach a second set point temperature, such as 32.5° C., and the infusate temperature 604 can begin to rise in temperature.

If the irrigate/drain mode of operation terminates when the patient temperature reaches a threshold temperature (e.g., 32.5° C.) or when the fluid reservoirs are empty, the system can go into pre-drain at step 4, to remove fluid until a preferred volume of infusate remains in the patient (e.g. 2 liters). Referring to FIG. 6, the volume of infusate in the patient can approach 2 liters in this mode.

At step 5, the system can go into an overshoot-stop mode of operation to warm the patient to a target temperature if the patient's temperature gets too low. Warming the fluid within the patient cavity can prevent the patient's core temperature from continuing to decline. For example, if the patient temperature goes below 32.5° C., the overshoot-stop mode can warm the patient back to 32.5° C. Referring to FIG. 6, the infusate temperature 604 can go up, which brings up the patient temperature 602.

At step 6, the system can go into a recirculate mode of operation, as described above. This will maintain the core temperature of the patient at the target temperature. As can be seen in FIG. 6, the infusate temperature 604, the patient temperature 602, and the infusate volume 606 can remain constant during recirculate mode. The recirculate mode of operation can be used in some embodiments to reach the second set point temperature of the patient after the initial fill and irrigate modes of operation have lowered the patient's core temperature to the first set point temperature. Reaching the second set point temperature at a rate slower than the first set point was reached is desirable to prevent overshoot of the patient temperature beyond a maintenance temperature (e.g., to cool the patient to a temperature lower than the maintenance temperature could cause patient harm). If the patient is cooling too rapidly towards the second set point temperature, there is risk that the patient will overshoot the second set point temperature. If the patient is cooling too slowly, the system will convert from recirculate to irrigate mode to ensure that the greatest cooling power is applied.

Next, at step 7 of flowchart 500, the infusate in the patient can be warmed until the patient reaches 36° C. Referring to FIG. 6, the infusate temperature 604 can go up, which brings up the patient temperature 602.

Finally, at step 8, the fluid can be removed from the patient during the drain mode of operation. The infusate volume 606 goes down to zero, which allows the patient temperature 602 to return back to 37° C.

FIGS. 5B and 6B illustrate yet another method of treatment and the treatment curve of patient core temperature vs. treatment time. In FIG. 5B, the fill and irrigate modes can be used, as described above, to achieve a first set point temperature (e.g., 32.5° C.) with fill mode and a second set point temperature (e.g., 34° C.) with the irrigate mode. Typical infusion rates are shown for each mode.

After reaching hypothermic temperatures (<34° C.), the system can enter maintenance mode. In maintenance mode, the system controls patient temperature according to a temperature/time curve. The curve can be prescribed or adjusted real time. The temperature/time curve can contain additional cooling, temperature maintenance sections, and warming sections. Transitions between sections may be based on time, or other conditions such as intracranial pressure, cavity pressure, core temperature, fluid volume infused, etc. A typical treatment temperature/time curve is shown in FIG. 6B. Both the rate of change of the patient temperature over time and the patient temperature may serve as input into the patient temperature control algorithm. The system drives the patient temperature towards the treatment curve by selecting the following controls: Mode (irrigate vs. recirculate), flow rate, infusate temperature (heat exchanger output). In one embodiment, the following rules can apply to maintenance mode:

TABLE 1

| Condition | Mode | Pump Speed | Peltier |
|---|---|---|---|
| Patient Temp ≧ 34° C. | Irrigate | Fast | 100% Cold |
| AND Unclog In | | | |
| never called | | | |
| <34° C. OR Unclog In called | Irrigate | Slow | 100% Cold |
| <33° C. and cooling slope | Recirculate | Slow | 100% Cold |
| similar to control curve. | | | |
| <33° C. and cooling slope | Recirculate | Slow | 50% Warm |
| too steep | | | |
| Patient at target temperature | Recirculate | Slow | OFF |
| Patient 0.5° C. below | Recirculate | Slow | 50% Warm |
| target temperature | | | |
| Patient < 32° C. | Recirculate | Slow | 100% Warm |

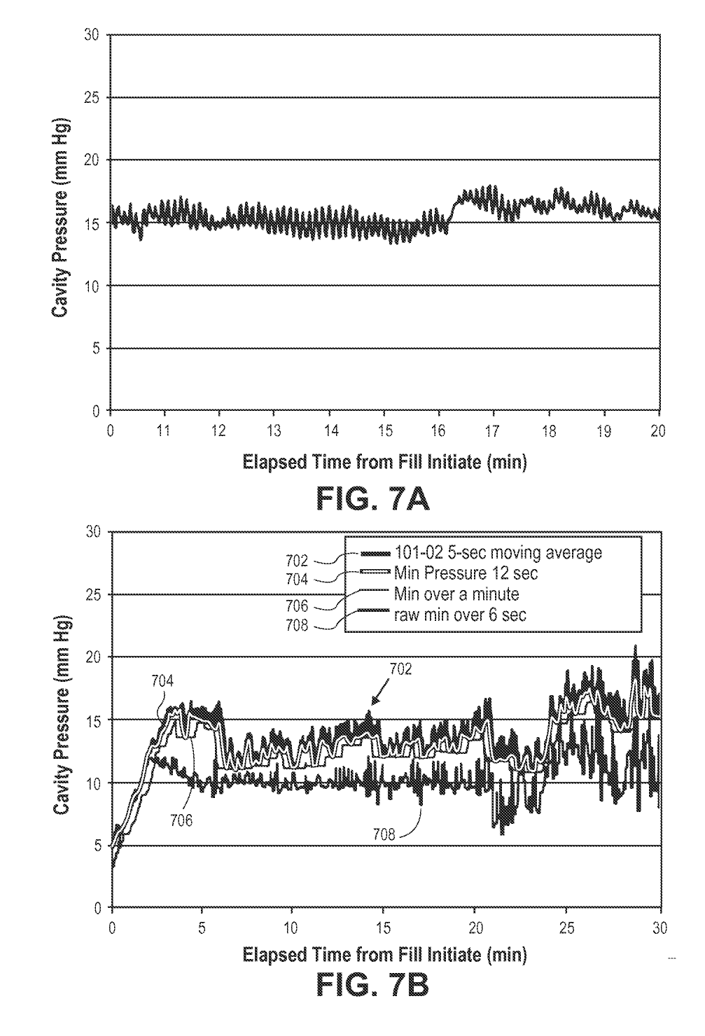

FIGS. 7A-7B show plots of the respiratory cycle during treatment described herein. By determining the therapy operating mode to use based on monitoring the respiratory cycle and cavity pressures, the device effect on pressure can be isolated from patient breathing, motion, and other artifacts. FIG. 7A shows a typical pressure signal that has been filtered with a 5-second moving average but still has respiratory artifacts.

FIG. 7B shows several signal conditioning options, including plot 702 which is the 5-second moving average of the pressure data. This averages out most of the respiratory cycle. Plot 704 is the minimum pressure over 12 seconds for plot 704. Plot 706 is the minimum pressure over a minute for the 5-second moving average pressure. Plot 708 is the minimum pressure over 6 seconds (approximately 1 breath) for the raw pressure signal. The benefit of conditioning the signal in this way is to isolate device effects from the patient effects on pressure. It can filter out artifacts from coughing, patient manipulation, and so forth on the pressure signal.

Another approach is based on the literature to determine pressure levels and durations that are considered safe in the peritoneal cavity. A look-up table such as the one below in tables 2 can be used.

TABLE 2

| Activity | Duration | Pressure |
|---|---|---|
| Cough | 1 second | 200 mm Hg |
| Cough | 5 seconds | 100 mm Hg |
| Exercycling | 10 seconds | 35 mm Hg. |
| Laparoscopic access | 5 minutes | 25 mm Hg |

Table 2 can translate into the following software parameters in Table 3:

TABLE 3

| Pressure | Duration |
|---|---|
| >200 mm Hg | 0 seconds (Immediate system fault) |
| 100 to 200 mm Hg | 1 second |
| 35 to 100 mm Hg | 5 seconds |
| 25 to 35 mm Hg. | 10 seconds |
| 15 to 25 mm Hg | 5 minutes |

The signal conditioning described in FIGS. 7A-7B and the lookup table in table 2 can be used together or separately, for example.

A method of attaching the LAS will now be discussed. Packaging that contains the LAS prior to attaching to the catheter and main unit includes layers that keep the various layers of LAS from tangling. The first packaging layer is the waste container 80, followed by the tubes that attach to the fluid reservoirs, followed by the sterile pouch 132, and finally the heat exchanger module 112. Each layer has a chip-board piece of paper with pictorial instructions on how to connect to the main unit 40.

Once Access to the patient has been achieved, a nurse or other medical practitioner can open the recirculation pouch, and the physician can pull out the patient lines with sterile hands. After removal of the recirculation cap, the patient lines can be connected to the catheter at the hub and the pressure device 102c of the catheter can be connected to the pressure sensors 120b at the hub. In the event that the user removes the recirculation cap prematurely in the patient preparation process, the system can detect a missing cap by a periodic leak down test whereby the patient lines are pressurized to a nominal pressure (15 psi for example) and the pressure is held for a set amount of time (5 seconds for example). This method can distinguish between cap present, cap missing, and catheter connected.

The display 44 may also provide indicators when the unit components are being connected. First, a picture of the system with status indicators (green solid light, red flashing light, etc) can show what components have been installed and what components are missing. Indicators can include the waste container being attached, pressure sensor connections, patient temperature connections, infusate temperature sensor connections, pump heads being closed, infusate reservoirs being attached, AC power being connected, tubing placed in pinch valves, refrigerator door closed, etc. A similar pictorial approach is used to remind the user of how to reconnect the system after a transport mode. Video tutorials can be played by the user to assist with the assembly of the LAS to the machine.

This indicator approach can be used when the system is being reloaded or detached. The graphics or indicators can show the user what components are to be removed and discarded. The system can also ensure that the user hangs fresh fluid reservoirs in the refrigerator prior to going into a standby mode.

A method of lifting the abdominal wall away from the peritoneal or other organs will now be discussed. This is commonly referred to as “tenting.” When access to the patient cavity has been gained with a threaded trocar, as described above, tenting can be accomplished by lifting directly on the threaded trocar once it is engaged in muscle. This provides clearance between the tip of the access device and organs when the tip enters the cavity. It also provides clearance for fluid entry to flow robustly into the cavity. Being able to pull up on the muscles requires having sufficient thread pitch and depth to engage muscle. Rotation of the access port from a vertical to a horizontal orientation can provide additional traction for tenting as well as provide a access path for the catheter to be inserted tangent to the peritoneal wall. This method is superior to the current practice of grasping the skin with towel clamps and pulling on the skin because pulling on the skin does not necessarily lift the muscle layer, it only expands the subcutaneous layer. With this method, it is possible to detect the different layers of tissue with the mobility of the distal end of the access device. In the subcutaneous tissue, the distal end of the access device can be very mobile (can be moved centimeters). Once engaged in muscle, the distal end of the access device does not move laterally. Typically, fluid can be added to the cavity entry indicator (fluid holder 65) once the access port is engaged in muscle. The muscle can provide a tight seal that prevents loss of fluid until the cavity has been entered.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

What is claimed is:

1. A method of inducing therapeutic hypothermia in a patient, comprising:
a) infusing a fill volume of hypothermic fluid into a patient cavity to cool the patient at a first cooling rate until reaching a first end condition;
b) after step a), removing hypothermic fluid from the patient cavity while infusing hypothermic fluid into the patient cavity to cool the patient to a first set point temperature at a second cooling rate; and
c) after step b), removing hypothermic fluid from the patient cavity while infusing hypothermic fluid into the patient cavity to cool the patient to a second set point temperature at a third cooling rate, the third cooling rate being slower than the second cooling rate.

2. The method of claim 1 wherein the first end condition comprises a sensed pressure in the cavity reaching a pressure limit.

3. The method of claim 2 wherein the pressure limit is greater than 14 mm Hg.

4. The method of claim 1 wherein the first end condition comprises infusing a fill volume of approximately 2 liters.

5. The method of claim 1 wherein the first end condition comprises infusing a fill volume of approximately 3 liters.

6. The method of claim 1 wherein the first end condition comprises infusing a fill volume of approximately 4 liters.

7. The method of claim 1 wherein the first end condition comprises infusing a fill volume of approximately 5 liters.

8. The method of claim 1 wherein the first end condition comprises infusing a fill volume of approximately 6 liters.

9. The method of claim 1 wherein the first set point temperature is approximately 34° C.

10. The method of claim 1 wherein the second set point temperature is approximately 32.5° C.

11. The method of claim 1 wherein the first cooling rate is approximately 12° C./hr.

12. The method of claim 1 wherein the second cooling rate is approximately 6° C./hr.

13. The method of claim 1 wherein the third cooling rate is approximately 1° C./hr.

14. The method of claim 1 wherein in step a), the hypothermic fluid is infused at a rate of approximately 1-2 liters per minute.

15. The method of claim 1 wherein in step b), the hypothermic fluid is infused and removed at the same rate.

16. The method of claim 1 wherein in step c), the hypothermic fluid is infused and removed at the same rate.

17. The method of claim 1 wherein in step b), the hypothermic fluid is infused and removed at a rate of approximately 0.1 to 0.3 liters per minute.

18. The method of claim 1 wherein in step c), the hypothermic fluid is infused and removed at a rate of approximately 0.1 to 0.15 liters per minute.

19. The method of claim 1 wherein the hypothermic fluid infused in step b) is colder than the hypothermic fluid infused in step c).

20. The method of claim 1 wherein the hypothermic fluid infused in step b) is a fresh hypothermic fluid from outside of the patient.

21. The method of claim 1 wherein the hypothermic fluid infused in step c) is a recycled hypothermic fluid from inside of the patient.

22. The method of claim 21 wherein the recycled hypothermic fluid is cooled prior to being re-infused into the patient.

23. The method of claim 1 further comprising detecting an obstruction, and reversing a flow direction of the hypothermic fluid to remove the obstruction.

24. The method of claim 1 further comprising:
d) draining the hypothermic fluid from the patient.

25. The method of claim 24 wherein in step d), the hypothermic fluid is drained at a rate of approximately 0.1 liters per minute.

26. The method of claim 1, wherein if the second cooling rate of step c) is below a desired rate, increasing the cooling rate.

27. The method of claim 1 further comprising adjusting the first and second cooling rates in real time by adjusting at least one of a pump speed, a cooling/warming power, or a fluid source based on a treatment parameter.

28. The method of claim 27 wherein the treatment parameter comprises a time of treatment.

29. The method of claim 27 wherein the treatment parameter comprises a cavity pressure.

30. The method of claim 27 wherein the treatment parameter comprises a core temperature.

31. The method of claim 27 wherein the treatment parameter comprises the fill volume.

32. The method of claim 1 wherein the fill volume is determined based on an amount of cooling energy needed to cool the patient.

33. The method of claim 2 further comprising:
d) infusing additional hypothermic fluid beyond the fill volume into a patient cavity and surpassing the pressure limit so as to cool the patient faster than the first, second, or third cooling rates; and
e) removing the additional hypothermic fluid after a pre-determined time period so the pressure limit is not surpassed beyond a tolerable limit.

34. The method of claim 1 further comprising maintaining therapeutic hypothermia in the patient with steps a)-c) for at least 24 hours.

35. The method of claim 1 further comprising warming the patient back to normothermic temperature.

36. The method in claim 35 wherein a rate of warming is controlled to a specified rate of warming.

37. The method of claim 35 wherein if the patient is warming too quickly, a cooler hyperthermic solution is circulated in the patient cavity to arrest a warming rate.

38. The method of claim 35 wherein if the patient is warming too slowly, a warmer hyperthermic solution is circulated in the patient cavity to increase a warming rate.

39. The method of claim 35 wherein if the patient is warming too quickly, a pump rate of hyperthermic solution can be lowered to arrest a warming rate.

40. The method of claim 35 wherein if the patient is warming too slowly, a pump rate of hyperthermic solution can be increased to increase a warming rate.

41. A method of inducing therapeutic hypothermia in a patient, comprising:
a) infusing a fill volume of hypothermic fluid into a patient cavity to cool the patient at a first cooling rate until reaching a first end condition; and
b) after step a), removing hypothermic fluid from the patient cavity while infusing hypothermic fluid into the patient cavity to cool the patient to a set point temperature at a second cooling rate.

42. The method of claim 41 wherein in step b), the infused hypothermic fluid is drawn from a remote fluid reservoir.

43. The method of claim 41 wherein in step b), the removing and infusing hypothermic fluid steps are performed at the same fluid flow rate.

44. The method of claim 41 wherein the fill volume is maintained at the same level during step b).

45. The method of claim 38 wherein the set point temperature is approximately 34° C.

46. The method of claim 38 wherein the set point temperature is approximately 32.5° C.
